(19) United States
(12) Patent Application Publication
(10) Pub. No.: US 2021/0193302 A1
(43) Pub. Date: Jun. 24, 2021

(54) OPTIMIZING PATIENT PLACEMENT AND SEQUENCING IN A DYNAMIC MEDICAL SYSTEM USING A COMPLEX HEURISTIC WITH EMBEDDED MACHINE LEARNING

(71) Applicant: GE Precision Healthcare LLC, Milwaukee, WI (US)

(72) Inventors: Andrew Day, Newton, PA (US); Bex George Thomas, Laguna Nigel, CA (US)

(21) Appl. No.: 16/721,219

(22) Filed: Dec. 19, 2019

(51) Int. Cl. G16H 40/20; G16H 10/60; G06Q 10/04; G06Q 10/06

(52) U.S. Cl. CPC G16H 40/20; G06Q 10/0633; G06Q 10/04; G16H 10/60

(57) ABSTRACT

Techniques are described for optimizing patient placement and sequencing in dynamic medical environment. In various embodiments, a method includes receiving current state information regarding a current state of a medical facility system in real-time, including operating conditions data regarding current operating conditions of the medical facility system and patient case data regarding active patient cases and pending patient cases of the medical facility system. The method further includes forecasting future state information for the medical facility system based on the current state information using a machine learning framework, including forecasted timeline information regarding future timing of workflow events of the active patient cases and pending patient cases. The method further includes employing a heuristic-based optimization mechanism to determine optimal reactive solutions regarding patient sequencing, patient placement and resource allocation based on the current state information, the future state information, defined rules of the medical care facility system, and one or more defined optimization criteria.

### TECHNICAL FIELD

This application relates to computer-implemented techniques for optimizing the sequencing and placement of patients in a dynamic medical system with shared and sub-specialized resources using a complex heuristic with embedded machine learning.

### BACKGROUND

Increasingly large hospitals and regional medical centers seek to optimize flow and throughput through procedural areas while providing safe and efficient care with higher and higher levels of utilization. One way to achieve efficiencies is to share resources between procedural modalities such as multiple surgical areas, interventional radiology (IR) procedure areas, catheterization laboratory (Cath Lab) procedure areas, and electrophysiology (EP) procedure areas. Although the concept of sharing staff and space across these modalities sounds simple, sub-specialization of staff, scheduling and transactional information technology (IT) systems/modules, and the frenetic pace and scale of operations make it very difficult for a human to optimally place and sequence arriving and transitioning patients in real time without some form of decision support.

### SUMMARY

The following presents a summary to provide a basic understanding of one or more embodiments of the invention. This summary is not intended to identify key or critical elements or delineate any scope of the different embodiments or any scope of the claims. Its sole purpose is to present concepts in a simplified form as a prelude to the more detailed description that is presented later. In one or more embodiments described herein, systems, computer-implemented methods, apparatus and/or computer program products are described that provide for optimizing the sequencing and placement of patients in a dynamic medical system with shared and sub-specialized resources using a complex heuristic mechanism with embedded machine learning.

According to an embodiment, a system is provided that comprises a memory that stores computer executable components, and a processor that executes the computer executable components stored in the memory. The computer executable components can comprise a reception component that receives current state information regarding a current state of a medical facility system in real-time over a course of operation of the medical facility system, wherein the current state information comprises operating conditions data regarding current operating conditions of the medical facility system and patient case data regarding active patient cases and pending patient cases of the medical facility system. The computer executable component can further comprise a forecasting component that employs a machine learning framework to forecast future state information for the medical facility system based on the current state information, wherein the future state information includes forecasted timeline information regarding future timing of initiation or completion of workflow events of the active patient cases and pending patient cases. The computer executable components further comprise an optimization component that employs a heuristic-based optimization mechanism to determine optimal reactive solutions regarding patient sequencing, patient placement and resource allocation based on the current state information, the future state information, defined rules of the medical care facility system, and one or more defined optimization criteria.

In various implementations, the forecasting component can further repeatedly (e.g., regularly or continuously) forecast and update the future state information based on the current state information as the current state information are received, and the optimization component can further repeatedly (e.g., regularly or continuously) determine and update the optimal reactive solutions based on the current state information as the current state information are received and the future state information as it is forecasted and updated. The computer executable components can further comprise a reporting component that provides the future state information and solutions information regarding the optimal reactive solutions as they are respectively forecasted and determined in real-time to one or more entities at the medical care facility system to facilitate managing operations of the medical care facility. In some implementations, the computer executable components further comprise a display component that displays the future state information and the solutions information in real-time via a graphical user interface tile at one or more devices associated with one or more entities.

In various embodiments, the medical facility system comprises a perioperative system where patients receive medical treatment in accordance with a defined perioperative workflow that involves movement of the patients to different procedural areas in association with the initiation or the completion of the workflow events, and wherein the optimization component determines how to sequence placing the patients in the different procedural areas in accordance with the defined perioperative workflow. With these embodiments, the one or more defined optimization criteria comprises minimizing delays between the workflow events and initiation of the pending patient cases. The one or more defined optimization criteria can also include criteria selected from a group consisting of: minimizing blocking of patients to required treatment, maximizing patient flow, minimizing costs, and minimizing surgeon wait time between procedures. For example, in some implementations, the current operating conditions data comprises bed status information identifying availability status of beds in the different procedural areas and staffing information identifying clinicians assigned to the beds. With these implementations, the optimization component can determine how to sequence placing the patients in the different procedural areas based on the forecasted timeline information, the availability status of the beds, the clinicians assigned to the beds, first defined rules regarding types of beds where the patients can be placed, and second defined rules regarding preferred clinician to patient ratios in the different procedural areas using a combinatorial optimization function that determines an optimal sequence for placing the patients that best achieves and/or balances one or more of the defined optimization criteria above.

In one or more implementations, the patient case data comprises status tracking information for the active patient cases indicating a current status of the active patient cases relative to the defined perioperative workflow and wherein the forecasting component forecasts the timeline information based on the current status of the active patient cases and historical state information regarding historical timing of the workflow events for historical patient cases under varying operating conditions of the perioperative system. With these implementations, the forecasting component comprises a case timeline forecasting component that forecasts the timeline information using one or more machine learning models trained on the historical state information, and wherein the case timeline forecasting component regularly updates the one or more machine learning models based on the current state information.

In some implementations, the future state information further includes forecasted demand information regarding forecasted demand for resources at the different procedural areas at different times over a defined upcoming timeframe, and wherein the resources include staff. With these implementations, the forecasting component can comprise a demand forecasting component that determines the forecasted demand information based on the current state information and the forecasted timeline information. The computer executable components further comprise a compliance monitoring component that determines whether available system resources at the different procedural areas at the different times comply with a resource constraint for the perioperative system based on forecasted demand, and an alert component that generates an alert regarding failure of the available system resources to comply with the resource constraint based on determination that the available system resources at a procedural area of the different procedural areas at a time of the different times, fail to comply with the resource constraint. The compliance monitoring component can also identify imbalances between the forecasted demand for the resources and available system resources at the different procedural areas at the different times, and the optimization component can further employ the heuristic-based optimization mechanism to determine the optimal reactive solutions based on the imbalances.

According to another embodiment, a system is provided that comprises a memory that stores computer executable components, and a processor that executes the computer executable components stored in the memory. The computer executable components can comprise a reception component that receives current state information regarding a current state of a perioperative system where patients receive medical treatment in accordance with a defined perioperative workflow that involves movement of the patients to different procedural areas in association with initiation or completion of defined workflow events, wherein the current state information comprises operating conditions data regarding current operating conditions of the perioperative system and patient case data regarding active patient cases and pending patient cases of the perioperative system. The computer executable component can further comprise a case timeline forecasting component that forecasts, based on the current state information and using a machine learning framework, timeline information regarding future timing of the initiation or the completion of the defined workflow events for the active patient cases and the pending patient cases. The computer executable component can further comprise an optimization component that employs a heuristic-based optimization mechanism to determine how to sequence placing the patients in the different procedural areas based on the current state information, the timeline information, defined rules of the perioperative system, and one or more defined optimization criteria.

In some embodiments, elements described in the disclosed systems can be embodied in different forms such as a computer-implemented method, a computer program product, or another form.

### DESCRIPTION OF THE DRAWINGS

FIG. 1 illustrates a block diagram of an example, non-limiting system for optimizing the sequencing and placement of patients in a dynamic medical system with shared and sub-specialized resources in accordance with one or more embodiments of the disclosed subject matter.

FIG. 2 presents a high-level overview of an example perioperative system in accordance with one or more embodiments of the disclosed subject matter.

FIG. 3 presents example patient placement prioritization rules of an example perioperative system in accordance with one or more embodiments of the disclosed subject matter.

FIG. 4 illustrates a high-level flow diagram an example, non-limiting process for optimizing the sequencing and placement of patients in a dynamic medical system using a continuous predictive-reactive scheme in accordance with one or more embodiments of the disclosed subject matter.

FIG. 5A illustrates a high-level flow diagram an example, non-limiting process for training and updating machine learning models configured to forecast future states of a dynamic medical system based on a current state of the dynamic medical system in accordance with one or more embodiments of the disclosed subject matter.

FIG. 5B illustrates a high-level flow diagram an example, non-limiting process for forecasting futures states of a dynamic medical system based on a current state of the dynamic medical system in accordance with one or more embodiments of the disclosed subject matter.

FIG. 5C illustrates a high-level flow diagram another example, non-limiting process for optimizing the sequencing and placement of patients in a dynamic medical system using a continuous predictive-reactive scheme in accordance with one or more embodiments of the disclosed subject matter.

FIG. 6 illustrates a block diagram of another example, non-limiting system for optimizing the sequencing and placement of patients in a dynamic medical system with shared and sub-specialized resources in accordance with one or more embodiments of the disclosed subject matter.

FIGS. 7A and 7B provide a chart that identifies forecasted resource demands and identified resource supply/demand imbalances for a perioperative system over an upcoming timeframe in accordance with one or more embodiments of the disclosed subject matter.

FIG. 8 presents an example dashboard for presenting to clinicians comprising real-time information regarding the current and forecasted state of a dynamic medical system and optimized patient placement and sequencing information in accordance with one or more embodiments of the disclosed subject matter.

FIG. 9 illustrates an example, high-level flow diagram of a computer-implemented process for optimizing the sequencing and placement of patients in a dynamic medical system in accordance with one or more embodiments of the disclosed subject matter.

FIG. 10 illustrates another example, high-level flow diagram of a computer-implemented process for optimizing the sequencing and placement of patients in a dynamic medical system in accordance with one or more embodiments of the disclosed subject matter.

FIG. 11 illustrates another example, high-level flow diagram of a computer-implemented process for optimizing the sequencing and placement of patients in a dynamic medical system in accordance with one or more embodiments of the disclosed subject matter.

FIG. 12 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Summary section or in the Detailed Description section.

The disclosed subject matter is directed to systems, computer-implemented methods, apparatus and/or computer program products are described that provide for optimizing the sequencing and placement of patients in a dynamic medical system with shared and sub-specialized resources using a complex heuristic with embedded machine learning. In various implementations, the dynamic medical system is a perioperative system in which patients receive medical treatment, (e.g., surgical procedures), in accordance with a defined perioperative workflow that involves movement of the patients to different procedural areas in association with the preoperative, interoperative and postoperative phases.

In one or more embodiments, a management system is provided that consumes real-time data feeds from IT platforms associated with various modality specific and phase specific components of the perioperative system and performs a complex optimization routine across the various the components as the state and context changes over the course of the day (e.g., as patients arrive early or late, add-ons/cancelations occur, schedules change, staff move between areas, etc.) to optimally place and sequence arriving and transitioning patients in real time. In this regard, the management system can output information that provides a specific sequence and placement of patients to minimize delay, maximize efficiency and satisfy a variety of rules and constraints. The overall optimization process employs a number of embedded sub-modules that consume real-time feeds and historical data and invokes machine learning algorithms which provide outputs that serve as inputs to a time-based optimization heuristic. Users can interact with the management system through a user interface (UI) in the form of a command center tile application. The management system also populates a simulator that forecasts future localized staffing supply/demand mismatches which can trigger a constraint under certain conditions which changes behavior of the sequencer unless over-ridden by the user.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

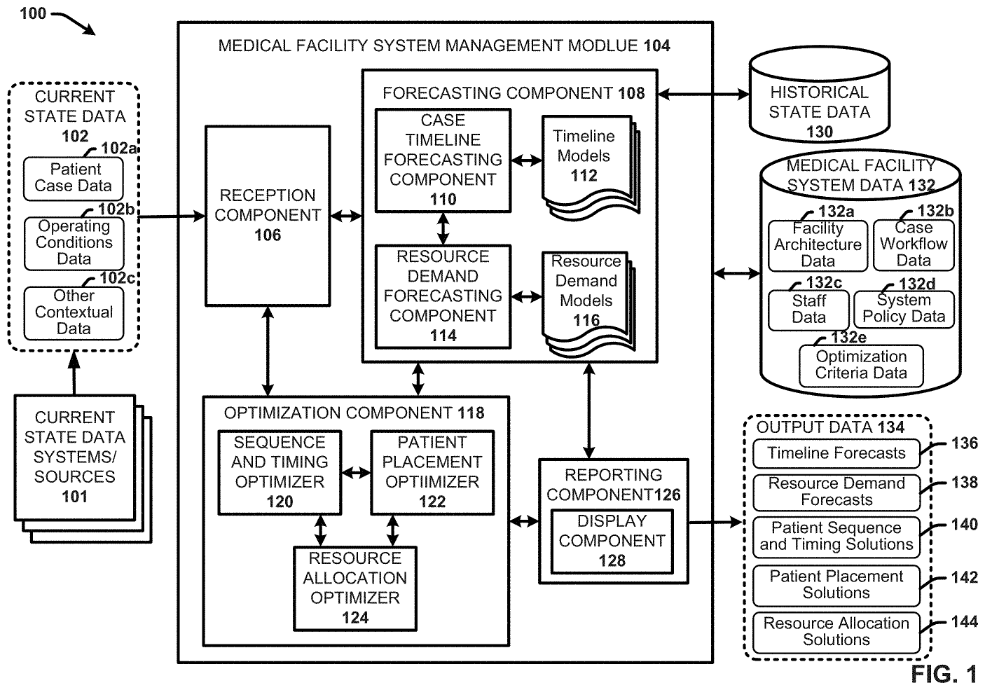

Turning now to the drawings, FIG. 1 illustrates a block diagram of an example, non-limiting system 100 for optimizing the sequencing and placement of patients in a dynamic medical system with shared and sub-specialized resources in accordance with one or more embodiments of the disclosed subject matter. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

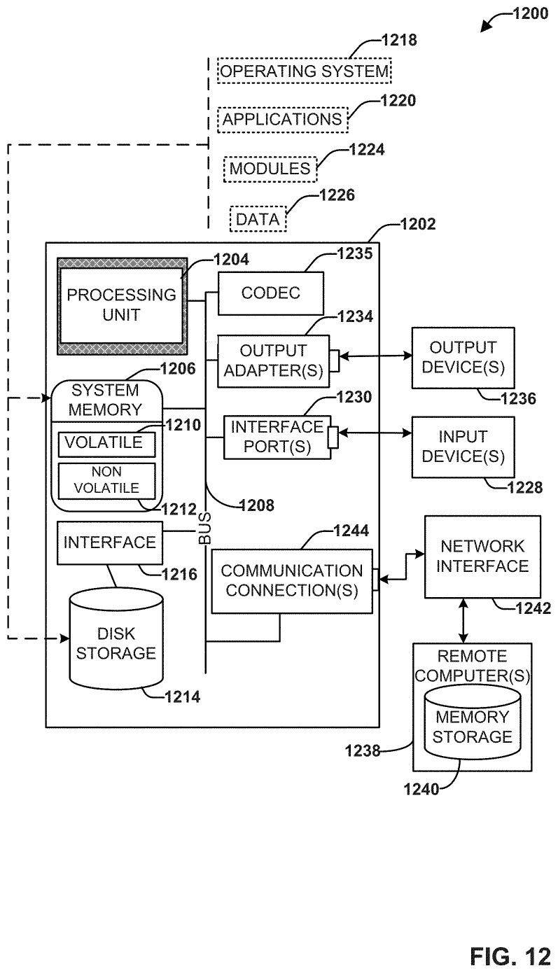

For example, system 100 includes a medical facility system management module 104 that can be and include computer/machine executable components. In the embodiment shown, the computer/machine executable components of the medical facility system management module 104 include a reception component 106, a forecasting component 108, an optimization component 118 and a reporting component 126. These computer/machine executable components (and other described herein) can be stored in memory (not shown) associated with the one or more machines (not shown). The memory can further be operatively coupled to at least one processor (not shown), such that the components (e.g., the medical facility system management module 104 itself, the reception component 106, the forecasting component 108, the optimization component 118, the reporting component 126, and other components described herein), can be executed by the at least one processor to perform the operations described. Examples of said and memory and processor as well as other suitable computer or computing-based elements, can be found with reference to FIG. 12, and can be used in connection with implementing one or more of the systems or components shown and described in connection with FIG. 1 or other figures disclosed herein.

In one or more embodiments, the medical facility system management module 104 provides real-time decision support regarding how to optimally place and sequence arriving and transitioning patients as they arrive and move through a dynamic medical facility system to facilitate optimizing the efficiency and quality of the medical care delivery process. In some embodiments, the medical facility system management module 104 can also provide other optimal reactive solutions in real time regarding allocation and utilization of shared resources (e.g., staff, beds, medical supplies and equipment, etc.) between different specialty areas/departments of the dynamic medical facility system to further facilitate optimizing the efficiency and quality of the care delivery process while minimizing costs.

As described herein, a real-time computer system can be defined a computer system that performs its functions and responds to external, asynchronous events within a defined, predictable (or deterministic) amount of time. A real-time computer system such as system 100 typically controls a process (e.g., the perioperative workflow), or a controlled system (e.g., a perioperative system) by recognizing and responding to discrete events within predictable time intervals, and by processing and storing large amounts of data acquired from the controlled system (e.g., current state data 102, historical state data 130, and medical facility system data 132). Response time and data throughput requirements can depend on the specific real-time application, data acquisition and critical nature of the type of decision support provided. In the regard, the term “real-time” as used herein with reference to processing and generating information by the medical facility system management module 104 refers to performance of these actions within a defined or predictable amount of time (e.g., a few seconds, less than 10 seconds, less than a minute, etc.) between reception of the current state data 102 by the reception component 106. Likewise, the term real-time as used with reference to reception of the current state data 102 refers to reception of the current state data 102 by the reception component 106 from the one or more current state data systems/sources 101 within a defined or predictable amount of time (e.g., a few seconds, less than 10 seconds, less than a minute, etc.) after the corresponding information is generated by and/or received by the one or more current state data systems/sources 101.

The dynamic medical facility system controlled and/or managed by the medical facility system management module 104 can include essentially any medical facility system with limited/fixed resources that provides medical treatment to patients in accordance with one or more defined workflows (or care pathways), wherein the timing of the workflows can be impacted by variable operating states/conditions of the dynamic medical system. For example, the dynamic medical facility system can include but is not limited to: a hospital, a specialized hospital unit, a surgery center, a specialized care provider facility (e.g., a clinic or office), an outpatient facility, an ambulatory services system, a nursing home facility, an imaging/diagnostic facility, a traveling/in-home patient care system, a rehabilitation provider system, and the like. These various types of medical facility systems often follow defined workflows for patient treatment that define tasks to be performed and rules regarding when, where and by whom the tasks are to be performed. For example, depending on the particular medical facility, the patient, and the treatment to be provided, the tasks can include clerical tasks (e.g., checking a patient in and obtaining relevant information from the patient), clinical tasks (e.g., performing a clinical exam/assessment, performing a medical procedure, administering medication, and other such clinical tasks involving providing medical care to and/or assessing the patient, etc.), procedural tasks involving moving or transitioning a patient to different areas of the facility, and the like.

In one or more embodiments, defined tasks in patient care workflows or pathways are referred to herein as workflow events. The timing of patient care workflows can encompass the timing of initiation and completion of the entire workflow (e.g., from patient arrival to patient discharge), as well as the timing of initiation and completion (or duration) of individual workflow events (e.g., timing of initiation and completion of a procedure, timing of administration of medication, timing of movement of the patient to the recovery unit, etc.). In this regard, depending on the type of medical facility system and the type of patient care workflows that are performed, a variety of variable operating states/conditions of the dynamic medical facility system can influence the timing of the workflows, such as changes in patient and staff scheduling (e.g., associated with cancelations, additions, staff members inability to arrive or work as scheduled, etc.), timing of arrival of patients, staff and other resources (e.g., ambulatory services, medical equipment/supplies, etc.), occurrence of medical complications, arrival of emergency patients, inefficient clinician performance (e.g., procedures taking longer than expected), occurrence of procedural errors, and a variety of other factors.

In various embodiments, the dynamic medical facility system managed/controlled by the medical facility system management module 104 comprises a perioperative system that provides medical treatment (e.g., surgical procedures) to patients in accordance with one or more defined perioperative workflows. For example, in various implementations, the one or more defined perioperative workflows can include the different phases of the perioperative period. The perioperative period refers to the period of time extending from when a patient goes into a hospital, surgery center, clinic, etc., for surgery until the time the patient is discharged. The perioperative period is generally defined by three distinct phases which include the preoperative phase, the interoperative phase, and the postoperative phase. In most surgical systems, every surgery, regardless of the type surgery, is broken down into these phases to differentiate tasks and establish who is responsible for overseeing and delivering each stage of care.

In many large hospitals and medical centers, the different phases and/or different parts of the phases of the perioperative process are carried out in different procedural areas which provide different types of resources depending on the patient's needs throughout the perioperative process. For example, in some implementations, the perioperative system managed by the medical facility system management module 104 includes a preoperative area where patients get prepped for surgery, the operating rooms and procedure rooms where surgeries are performed, and the post-anesthesia care unit (PACU) where patients are monitored just out of surgery. In some implementations, perioperative systems also include a post-operative recovery area where patients are transitioned to from the PACU for recovery if not sent home (or to another inpatient facility). These different areas of the perioperative system are generally associated with different types of staff (e.g., clinicians, technicians, etc.) and equipment that are tailored to the workflow events that are performed at the respective areas and the associated patient care needs. For example, the PACU often includes sophisticated equipment and staff that is proficient and experienced in the subtleties of critical care immediately following surgery. In this regard, as a patient moves though the perioperative system, the patient is often moved to the different procedural areas and treated by different specialty staff trained to perform specific roles relevant to the patient's needs at different phases in the perioperative process. Perioperative systems often follow defined workflow rules and policies that control where (e.g., procedural areas) and when a patient is placed throughout their perioperative process, which can vary based on their needs, procedures being received, and the like.

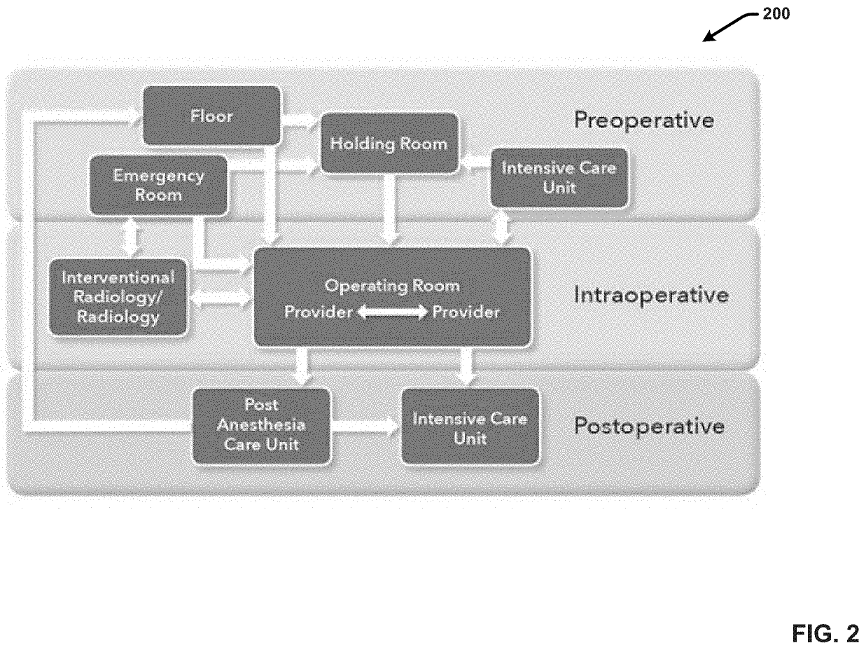

For example, FIG. 2 presents a high-level overview of an example perioperative system 200 in accordance with one or more embodiments of the disclosed subject matter. In some implementations, the example perioperative system 200 can be part of and/or attached to a hospital or clinic. The example perioperative system 200 includes a variety of different types of floors, rooms, and units, that are respectively associated with the different phases of the perioperative process. For example, in the embodiment shown, one or more aspects of the preoperative phase can be performed at the floor area, the emergency room (ER), a holding room, and/or the intensive care unit (ICU). In many hospitals, the “floor” area refers to an area, unit, bed etc., where a patient is placed when the patient does not require close monitoring. The interoperative phase includes the operating room (e.g., which can be subdivided into different specialty operating rooms), and the IR room, and the postoperative phase includes the PACU as well as the ICU. In accordance with example perioperative system 200, depending on a patient's needs and context, the patient may begin the preoperative phase at the floor, ER, or ICU. The patient may then be moved to the holding room prior to entering the interoperative phase areas (e.g., the operating room and/or the IR room) or moved directly from the floor, ER or ICU to the interoperative phase areas. From the operating room, the patient can be transferred to the PACU or ICU if necessary. Following recovery in the PACU, the patient will then be moved back to the floor for post-op recovery.

It should be appreciated that the various rooms, units, areas and patient flow pathways of the perioperative system 200 are merely exemplary and that the disclosed techniques can be applied to a variety of perioperative system architectures and flow pathways. For example, in some embodiments, the perioperative system managed by the medical facility system management module 104 can include a plurality of different preoperative areas, a plurality of different interoperative areas, and/or a plurality of different postoperative areas. In this regard, the different areas can correspond to different floors, different units, different rooms, different pods, different bays, different beds or the like. The different areas (e.g., floors, units, rooms, pods, bays, beds, etc.) within the perioperative, interoperative, and/or postoperative environments can further be associated with different features, functionalities and/or services. For example, the different areas can be tailored to different types of patients with different medical care needs or levels of acuity, tailored to different types of procedures, include different types of medical staff (e.g., with different specialties and credentials), include different types of beds, medical equipment, supplies, and the like. For example, in some embodiments, the perioperative system can be subdivided by surgical modality, wherein he surgical modality refers to a specific type of surgical procedure. For instance, in some implementations, the perioperative system can include one or more preoperative, interoperative and/or postoperative areas associated with general operating room procedures; one or more preoperative, interoperative and/or postoperative areas associated with specialty operating room procedures; one or more preoperative, interoperative and/or postoperative areas associated with IR procedures; one or more preoperative, interoperative and/or postoperative areas associated with Cath Lab procedures; and one or more preoperative, interoperative and/or postoperative areas associated with EP procedures.

With reference again to FIG. 1, in various embodiments, the medical facility system management module 104 can provide real-time decision support for managing and coordinating the flow of patients through a perioperative system (e.g., example perioperative system 200) as patients arrive, schedules change, staff move between areas and add-ons/cancelations occur, and the like. In this regard, in large hospitals and medical centers, there is a need for controlling or managing the perioperative process, as the upstream and downstream flow of patients in preoperative and post-operative units can affect the care of the patients and the overall surgical outcomes. For example, it is critical to manage when the patients start getting prepped for surgery, how much time they should be under anesthesia, when they should move from one stage to another, and the like, to ensure optimal patient flow, to ensure required care is provided to patients at the right time, to minimize complications, and to minimize costs associated with various delays. Although many perioperative systems schedule patient procedures to be performed for the day before the day begins, a plethora of factors can change over the course of the day that require the care delivery team to reschedule patients, reassign patients to different rooms, reprioritize patients, reallocate resources to the patients, and the like. For example, with respect to a perioperative system that begins a day with a set schedule of patient cases to be completed (including scheduled timing of the cases, clinicians assigned to the cases, etc.), over the course of the day patients may arrive early, late or not at all, emergent patients may be added, patients may experience complications that can result in the patient requiring additional procedures and/or unexpected care needs, staff availability may change, medical supplies and/or equipment may not be available as expected, procedures may take longer than expected, and a variety of other factors may occur.

In this regard, the medical facility system management module 104 can monitor and evaluate state of dynamic medical facility system in real-time based on the current state data 102, and using machine learning and heuristic based analytics, determine or facilitate determining how to react to these types of dynamic conditions over a course of operation of a medical facility system in a manner that optimizes the overall efficiency and quality of care. For example, with respect to a perioperative system, the medical system management module 104 can determine how to sequence (or prioritize) incoming and transitioning patients, and determine where (e.g., specific areas, rooms, units, bays, pods, beds, etc.) and when to place patients throughout their perioperative journey using shared resources (e.g., staff, areas, beds, medical equipment, etc.) between different specialty and sub-specialty areas (e.g., associated with different surgical modalities) based in part on the defined workflow rules and dynamic/variable conditions of the perioperative system. In this regard, the medical facility system management module 104 can manage and coordinate the upstream and downstream flow of different patients through the perioperative system in view of defined policies, rules and constraints of the perioperative system (e.g., provided by the medical facility system data 132) to achieve and/or balance one or more system performance objectives. In various embodiments, the one or more performance objectives can include, but are not limited to: minimizing delays between perioperative workflow events (e.g., prepping for surgery, prepping for anesthesia, initiating anesthesia, movement from one perioperative phase of care to the next, etc.), maximizing patient flow/throughput (e.g., frequency of completed cases), minimizing blocking to required care, minimizing costs, and minimizing surgeon idle time (e.g., wait time) between procedures.

To support such operations, the medical facility system management module 104 consumes real-time data feeds providing current state data 102 regarding the current state of the medical facility system (e.g., a perioperative system), historical state data 130 for the medical facility system (and/or one or more similar medical facility systems), and medical facility system data 132 describing constraints, rules/policies and objectives (e.g., embodied in the optimization criteria data) of the dynamic medical facility system.

In this regard, the current state data 102 can include a variety of information regarding the patients and operating conditions of the dynamic medical facility that vary over a course of operation of the dynamic medical facility (e.g., from minute to minute, hour to hour, day to day, etc.). For example, the current state data 102 can include information regarding current and pending patient cases of the medical facility system, including case status information with tracking of progression of workflow events, current patient status, and the like. The current state data 102 can also include information current operating conditions of the medical facility system, such as current bed status and availability, current patient and staff locations, current staff assignments and tasks being performed, and the like.

In the embodiment shown, the current state data 102 is grouped into three categories, including patient case data 102a, operating conditions data 102b and other contextual data 102c. The patient case data 102a can include information regarding current and pending patient cases of the medical facility system. In this regard the terms “patient case” and “case” as used herein refers to a specific patient and a specific course or type of medical care or treatment prescribed for the patient. For example, with respect to a perioperative system, a patient case can refer to a patient and the surgical procedure or procedures prescribed for the patient. Unless otherwise stated, all patient cases involve the performance of a defined workflow, which can vary based on the type of case, the patient, and other factors. In various implementations, information describing the defined workflows for the patient cases can be provided in medical facility system data 132 (e.g., as case workflow data 132b).

In various embodiments, a patient case is referred to as an “active” patient case” if the defined workflow for the patient case has been initiated as defined by occurrence of a defined initial event in the workflow. For example, in embodiments in which the dynamic medical facility system is a perioperative system, a patient case can be classified as an active patient case if the perioperative process or period for that patient has begun. In some implementations, the perioperative process or period can be considered initiated at the time the patient arrives and checks-in the perioperative facility and/or a defined preoperative arrival area for the patient. In other implementations, the perioperative process or period can be considered initiated based on occurrence of another defined event that marks the beginning of the perioperative workflow, such as movement of the patient to a preoperative area to begin prepping the patient for surgery.

As described herein, a patient case can be classified as “pending” if the defined workflow for the patient case patient has not been initiated, as defined by non-occurrence of a defined initial event in the workflow that marks the start of the workflow. For example, with respect to perioperative cases, pending patient cases can include scheduled patient cases and/or emergent patient cases (e.g., newly added/received patient cases) that have not been initiated. In this regard, in various embodiments, the perioperative system can include a system that schedules patient cases to be performed at defined dates and/or times. For example, many surgical centers schedule patient surgeries, days, weeks and/or months in advance. Upon the start of an operational time frame for the surgical center (e.g., the start of the day), those scheduled cases that have not yet been initiated can be considered pending cases, including the first case scheduled for the day and any cases scheduled to be performed in the future (e.g., that day, the next day, the next week, the next month, etc.). A patient case can be considered completed when the defined workflow for the case has been completed, as defined by the completion of a final workflow event for the defined workflow (e.g., discharge, or another defined event). For example, with respect to perioperative workflows, a patient case can be classified as completed in response to movement of the patient from PACU to recovery, in response to discharge, or based on occurrence of another defined event.

In various embodiments, for all patient cases (active and pending) managed by the medical facility system management module 104, the patient case data 102a can include information that identifies or indicates the patient, the case (e.g., via a case number or another identifies), and the type of the case and/or procedure or procedures involved. In some implementations, the patient case data 102a can also identify and/or describe the workflow to be followed for the case, a priority level of the case, the physician/clinician(s) assigned to the case, and any defined scheduling information for the case (e.g., regarding a scheduled time/date of the case). In other implementations, this information can be looked up in the medical facility system data 132 based on information identifying the patient and/or the case number. In some implementations, the reception component 106 can collect, extract or otherwise receive this type of patient case data from one or more case scheduling systems, clinical ordering systems, physician notes, case manager notes, and the like. For example, with respect to a perioperative system, the one or more case scheduling systems can include a unified scheduling system for the entire perioperative system, and/or individual scheduling systems for different procedural departments (e.g., grouped by modality or another factor). In this regard, the reception component 106 can receive, extract or otherwise collect information from the one or more case scheduling systems as the cases are scheduled and entered into the one or more scheduling systems. In some implementations, as the optimization component 118 determines changes to patient scheduling throughout the day with respect to patient sequence and timing 140 and/or patient placements 142, this information can be updated in the case scheduling systems and reflected in the current state data 102. The current state data 102 can also identify changes in case scheduling as they occur in real-time over the course of operation of the medical facility system as new cases are added, cases are canceled, rescheduled and/or the like. Accordingly, the reception component 106 can regularly or continuously receive updated information (e.g., in real-time) regarding what patient cases are scheduled for performance at the medical facility system within an upcoming timeframe (e.g., in the next hour, in the next 12 hours, the next 24 hours, the next 48 hours, the next week, etc.).

In addition, for active patient cases, the patient case data 102a can include real-time status information regarding the progress of the case, including what's currently happening to the patient, where the patient is located, who is currently treating/attending to the patient, and the like. For example, for active patient cases, the patient case data 102a can include a variety of real-time status information, including but not limited to: information regarding their current workflow status in their course of treatment (e.g., in surgery, in recovery, etc.), workflow events currently being performed (e.g., procedures being performed, medications being administered, movement of the patient from location A to location B, etc.), timing of workflow events (e.g., timing of initiation and/or completion of the workflow events and procedures), staff currently involved in the patient's care (e.g., physicians, nurses, technicians, etc.), information regarding the patient's current physiological and/or medical status (e.g., stable, under anesthesia, awake, etc.) and the like. The patient case data 102a can also include real-time information regarding changes in a patient's status, condition, diagnosis, workflow (e.g., new procedures/tasks to be performed), adverse reactions, and the like. In this regard, in embodiments in which patient cases follow defined workflows that include defined workflow events (e.g., procedures to be performed, tasks to be completed, etc.) to be performed in a defined order, the current state data 102 can track the progression of the workflow events and identify relevant information regarding the patient and the current workflow event underway.

For example, in implementations in which the defined workflows involve progression of the patient through the perioperative phases in sequential order (e.g., the preoperative phase, followed by the interoperative phase and the postoperative phase), the case status tracking information can identify the current phase that the patient is in. For instance, if a patient is currently in the interoperative phase (e.g., under surgery), the case status tracking information can indicate that patient “John Doe” or case number “123” is currently in the interoperative phase. Although this example reflects tracking workflow progression through the three perioperative phases, it should be appreciated that the level of granularity of tracked workflow events and the specific workflow events that are tracked can vary. The tracked case status information can not only indicate a current status of a patient's case relative to a defined workflow, but further track the timing of workflow events, including timing of initiation, completion and/or duration of the respective workflow events. For example, the tracked case status information can indicate that a patient is currently in PACU and has been there for N minutes. In this regard, the tracked case status information can include timestamps that reflect when certain workflow events are initiated and/or completed and/or indicate the duration of time a workflow event has been in progress. The tracked case status information can also include many other trackable parameters related to a patient's course of care, including information regarding patient location, patient physiological status, staff involved in the patient's care, medical equipment/supplies involved in the patient's care, and the like.

The reception component 106 can regularly and/or continuously receive the various types of case status information discussed above from a variety of current state data systems/sources 101 in real-time. For example, the reception component 106 can receive this status information from one or more from case status tracking systems, patient location tracking systems/devices, patient vital signs monitoring systems, medical monitoring devices associated with the patients, and the like. In some implementations, these tracking and/or monitoring systems/devices can include systems/devices configured to receive and report user input in real-time explicitly identifying and/or indicating such status information (e.g., regarding the patient's active case status, location, workflow event progress, staff involved, etc.). These tracking and/or monitoring systems/devices can also include intelligent systems/devices configured to determine and report various real-time status information without or with minimal manual input based on analysis of sensor/device captured data signals. For example, these tracking and/or monitoring systems/devices can also include systems/devices configured determine information regarding what's currently happening to the patient (e.g., workflow events), where the patient is currently located, who is currently treating/attending to the patient, and the like based on analysis of a variety of captured data, including but not limited to: image data (e.g., using object recognition and/or other image pattern recognition technologies), audio data (e.g., using speech to text and natural language processing NPL), motion sensor data (e.g., using motion pattern recognition technology), radio frequency (RF) data, temperature sensor data, light sensor data, infrared (IR) senor data, biometric sensor data, and the like.

The current state data 102 can also include operating conditions data 102b regarding the current operating conditions and/or context of the dynamic medical facility system. For example, in implementations in which the dynamic medical facility system is a hospital, a perioperative system or a similar type of system, the operating conditions data 102b can include information regarding but not limited to: current occupancy levels, current bed availably, current bed status (e.g., occupied, clean or dirty), number of patient waiting for beds, staff availability, staff location, staff activity, supply/instrument availability and location, equipment status and location (e.g., and the like. In this regard, with respect to a perioperative system wherein patients move to different procedural areas and beds over the course of their perioperative workflow, the operating conditions data 102b can include current bed status information identifying the availability status of beds in the different procedural areas, including where they are located, type of bed (e.g., in systems which have different types of beds for different types of medical needs), and whether the bed is occupied, available, dirty, and the like. In some implementation in which a bed is occupied, dirty or otherwise unavailable, the operating conditions data 102b can also indicate an expected time when the bed will become available. Similarly, the operating conditions data 102b can indicate the current status of procedure areas (e.g., operating rooms), including whether the are available, in-use, being cleaned/sterilized, etc. The current operation conditions data 102b can also include information regarding the current status of equipment (e.g., in use or available, being cleaned, dirty, etc.).

The operating conditions data 102b can also include information regarding staff, including the identities of staff currently working and/or available, where they are currently located, what they are currently doing, where they are currently assigned, and the like. For example, the current staff information can identify the staff (e.g., nurses, physicians, technicians, etc.) currently assigned to specific procedure areas and/or bed, cases, patients and the like, and their scheduled work hours/time-frame (e.g., nurse Amy N. is assigned to postoperative room number 152 until 6:00 pm). The current staff information can also identify tasks and/or activities currently being performed by and/or assigned for performance by specific staff members. In this regard, the activity information can identify or indicate whether a staff member is available, busy, with a patient, when they will be available to perform another task and the like. In another example, with respect to surgeons in particular, the staff data can indicate a current status/activity of a surgeon, including whether they are sterilized and ready (and waiting) to perform surgery, whether they are currently in surgery, whether the surgeon is currently preparing (e.g., being re-sterilized), etc. In various embodiments discussed in greater detail infra, information regarding current surgeon activity can be used by the optimization component 118 to facilitate determining how to manage operations of the medical facility system in real-time (e.g., with respect to patient placement, sequence and timing and resource allocation) to minimize surgeon idle time (e.g., wait time) between procedures. In some implementations, the operating conditions data 102b can also include information regarding the current staff fatigue level, stress level and the like (e.g., captured via one or more medical monitoring/biofeedback devices).

The operating conditions data 102b can also include known scheduling informaiton for staff within an upcoming timeframe, including who is scheduled to work, scheduled assignments (e.g., to patients, cases, rooms, beds, etc.), and when (e.g., timing). For example, with respect to a perioperative system, the staff scheduling information can identify surgeon schedules (e.g., including cases to perform and scheduled time/date of the cases) for the remainder of their current shift, for the next 24 hours, for the next 48 hours, for the next week, etc. In this regard, the staff scheduling information can include staff scheduling information for a defined upcoming timeframe (e.g., for the current shift, over the next hour, over the next 12 hours, over the next 24 hours, over the next 48 hours, over the next week, etc.), including who is scheduled to work and when (for what time frames), and where they are assigned (e.g., if assigned to a specific location, patient and/or task). As discussed in greater detail with reference to the optimization component 118, in some implementations, staff scheduling, and assignments can be changed and updated in real-time to facilitate optimizing operations at the medical facility system based on a current and/or forecasted state of the medical facility system. Such changes in staff scheduling and assignments can also be reflected in the current operating conditions data 102b.

The current state data 102 an also include various other type of contextual data 102c associated with the dynamic medical system that can or has been shown to historically effect and/or correlate to (e.g., either directly or indirectly) the operations of the medical facility system with respect to case workflow timing, supply/demand for resources, patient occupancy levels, bed availability levels, reception of emergent patients, patient cancelations and delays, patient complications, and the like. For example, in some implementations, the contextual data 102c can include information regarding time of day, day of week/year, localized events or conditions at the perioperative system or hospital in which the perioperative system is connected (e.g., emergency or crisis scenarios, disease outbreaks), local events associated high influx of patients, etc.) and the like.

The historical state data 130 can provide historical state information for the dynamic medical facility system (and/or one or more similar medical facility systems) collected over time providing detailed information regarding performance of historical patient cases and their workflows under varying operating conditions and contexts of the dynamic medical facility system. For example, the historical state data 130 can provide historical performance metrics regarding timing of workflows (e.g., including timing of discrete workflow events and overall timing of the workflows), patient outcomes, demand on resources and the like for different types of cases, patients, procedures, surgeons, workflows, etc. under various operating conditions/contexts. In another example, the historical state data 130 can include historical performance metrics for individual staff with respect to timing of performance of specific tasks/procedures (e.g., surgeon Nathen. J generally completes procedure xyz of patients with this level of severity in X time), quality of care, complication rate, etc. In this regard, in various embodiments, the historical state data 130 can include historical sets of the current state data 102 that were collected and aggregated for the dynamic medical facility system in the past. In this regard, the historical state data 130 can include the same (or similar) parameters included in the current state data 102 yet previously collected for past patient cases under past operating conditions of the dynamic medial facility system. In other implementations, the historical state data 130 can include historical state data collected for one or more similar medical facility systems.

The medical facility system data 132 can include variety of static and/or semi-static information about the dynamic medical facility that is managed by the medical facility management system module 104 that reflect optimization objectives of the dynamic medical facility system and known constraints. For example, in various embodiments, the medical facility system data 132 can include (but is not limited): facility architecture data 132a, case workflow data 132b, staff information 132c, system policy data 132d, and optimization criteria data 132e.

The facility architecture data 132a can include information regarding the physical structure, resources and layout of the dynamic medical facility system. For example, the facility architecture data 132a can include information identifying the different areas, wings, units, rooms, bays, pods, etc. of the medical facility and the specific classification or type of the respective areas, wings, units, rooms, bays, pods, beds etc. (e.g., floor, holding room, ICU, ER, OR, PACU, etc.). The facility architecture data 132a can also include informaiton identifying the number and type of beds associated with the different areas, wings, units, rooms, bays, pods, etc. The facility architecture data 132a can also include informaiton regarding medical equipment and supplies associated with the in the respective areas, wings, units, rooms, bays, pods, beds, etc. In some implementations, the facility architecture data 132a can include floorplan data that provides a floorplan of the medical facility, including information identifying relative locations of the respective areas, wings, units, rooms, bays, pods, beds, etc. For example, with respect to a perioperative system, the facility architecture data 132a can identify the different preoperative, interoperative and postoperative procedural areas, identifying specialty classifications of the respective areas (e.g., IR room, general OR room, OR room for prenatal patients, Cath Lab room, etc.), identify the relative locations of these different areas, identify the number and types of beds associated with the different areas, include information regarding the medical equipment and supplies located in the different areas, and the like.

The case workflow data 132b can include information that defines one or more workflows for patient cases that are performed at the dynamic medical facility system. The defined workflows can include information regarding the specific workflow events to be performed as well as rules regarding when, where, how and by whom the respective workflow events are to be performed. For example, the case workflow data 132b can including information identifying specific clinical and non-clinical tasks to be performed and include required (or preferred) timing information regarding sequence/order for performing the events, duration of the respective events, timing between events, and other conditions related to patient status, recovery time, etc., that c and the like. The case workflow data 132b can also include required or preferred location rules regarding where certain workflow events are to be performed (e.g., area, wing, room, bed, bay, pod, etc.), who can perform the workflow events (e.g., clinician type and/or required clinician credentials). The case workflow data 132b can define general workflows for all patients, as well different workflows for different types of cases, patients, procedures, diagnosis, clinicians, and the like. For example, in some implementations, all patient cases performed at the dynamic medical facility system can follow the same general workflow. For instance, in some implementations in which the dynamic medical facility system is a perioperative system, the general workflow can include a general perioperative workflow that involves performance of defined preoperative, interoperative and postoperative workflow events. For example, in one implementation, the perioperative system can require all perioperative patients to follow a care pathway that begins in a preoperative area, moves to a perioperative area, then to PACU and then to a postoperative recovery area. In other implementations, the case workflow data 132b can define different workflows for different types of surgical procedures, different patients (e.g., grouped by age or another demographic factor, level of acuity, severity, comorbidity, medical history, etc.).

The staff data 132c can include known information about the different staff (e.g., clinicians, nurses, physicians, technicians, administrative workers, etc.) of the dynamic medical facility system. In this regard, the staff data 132c can include information identifying the respective staff members (e.g., by name or another unique identifier) and their job title or role. The staff data 132c can also include information regarding their different qualifications, capabilities, authorizations for performing certain workflow events, skill levels, proficiency levels, performance levels, and the like. For example, the staff data 132c can identify specialty qualifications of nurses relevant to caring for different types of patients' and/or providing different types of clinical care/treatment in the perioperative environment. The staff data 132c can also include (but is not limited to), compensation information identifying the compensation schedule/salary of the workers, their demographic information (e.g., gender, age, ethnicity, etc.), home address/location, their contact information (e.g., phone number, email address, social media connections, etc.) and the like.

The system policy data 132d can any additional system policies regarding, rules, restrictions, protocols, rewards/penalties, etc., of the dynamic medical facility system that control or influence sequencing, timing and placement of patients and allocation of resources (e.g., including staff and other resources). For example, in various implementations, the system policy data 132d can include polices/rules regarding prioritization of patient cases. For instance, the system can assign different priority ranks to different types of cases/procedures, such that higher priority ranking cases are prioritized for performance before lower prioritized cases. The system policy data 132d can also include rule/policies regarding patient placement with respect to physical locations (e.g., floors, units, rooms, pods, bays, beds, etc.), and staff. For example, different areas of the perioperative system can provide different types of resources (e.g., different bed types) and/or serve different patient care needs. In this regard, the system policy data 132d can define hierarchical placement preferences for placing patients/cases to specific areas/beds and/or clinicians based on their priority level, level of acuity, procedure type, or another factor, at different phases of the perioperative process (e.g., pediatric patients should be placed in PACU 1, then PACU 2 if no beds in PACU 1 are available).

The system policy data 132d can also include resource information regarding restrictions or requirements of resources distribution and utilization. For example, in some implementations, the system policy data 132d can define required nursing to patient ratios, which can vary by patient, case type, physical location/area, (e.g., unit, room, pod, etc.), perioperative phase, or another factor. For instance, in the system policy data 132d can require PACU areas 1 and 2 have a nursing ratio of 2:1 (e.g., two patients for each nurse), while PACU areas 3 and 4 have a nursing ratio of 3:1. The system policy data 132d can also include information defining type and amount of resources to be used for certain tasks (e.g., workflow events), amount of resource to be made available for certain tasks, and the like. The system policy data 132d can also include information regarding time requirements for performance or workflows, specific workflow events, specific tasks, etc. (e.g., maximum and minimum durations for the workflows, tasks, etc.). In another example, the system policy data 132d can also define provide rules or regulations regarding qualifications of workers for performing certain tasks (e.g., required skill set), treating specific patients/cases, and the like.

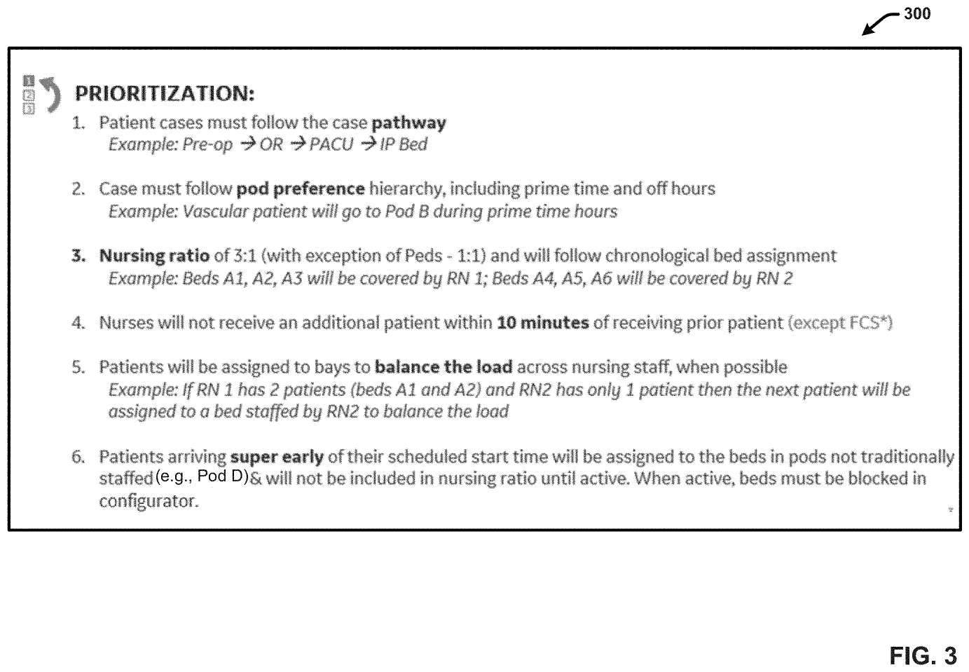

FIG. 3 presents example patient placement prioritization rules 300 of an example perioperative system in accordance with one or more embodiments of the disclosed subject matter. These patient placement prioritization rules 300 provide an example of the type of placement rules that can be defined by the system policy data 132. In the example, shown, six prioritization rules are provided. The first rule requires all patient cases to follow their defined case pathway (or workflow), which can be defined in the case workflow data 132b. This can vary based on the type of case, the patient, the procedure etc. In the embodiment shown, one example case pathway is exemplified which can be for all perioperative patients for instance (e.g., Pre-op to OR, to PACU to IP bed). The second rule regards pod preference hierarchy and indicates that a defined pod preference hierarchy for cases in prime time and off time hours is in place which must be followed accordingly. For example, in this context, a “pod” can refer to a specific preoperative area and/or postoperative area such that the preoperative area and/or postoperative area have more than one pod (e.g., the preoperative area could be divided into Pods A, B, C and D, and/or the postoperative area could be divided into Pods, E, F and G). In the embodiment shown, one example is provided indicating that “Vascular patients will go to Pod B during prime-time hours.”

The third rule provides a nursing assignment rule and states that a nursing ratio of 3:1 (e.g., one nurse for every three patients) is required for all patients except pediatric patients (which requires a nursing ratio of 1:1), and further states that nurse assignments will follow a chronological bed assignment order. For example, assuming a Pod A or another defined procedural area has several beds respectively identified as bed A1, bed A2, bed A3 and so on, in accordance with the 3:1 nursing ratio, each nurse will cover three chronological beds (e.g., Beds A1, A2 and A3 will be covered by RN 1; Beds A4, A5, and A6 will be covered by RN 2, and so on). The fourth rule states that nurses will not receive an additional patient within 10 minutes of receiving a prior patient.

The fifth rule states that patients will be assigned to bays to balance the load across nursing staff, when possible. For example, if RN 1 has 2 patients (in beds A1 and A2) and RN 2 has only 1 patient, then the next patient will be assigned to a bed staffed by RN 2 to balance the load. According to this example, a “bay” corresponds to a grouping of beds assigned to a single nurse (e.g., Beds A1, A2 and A3 assigned to RN 1 would correspond to a single bay). The sixth rule states that patients arriving super early (e.g., which can be relative to a defined threshold) of their scheduled start time will be assigned to beds in the pods not traditionally staffed (e.g., Pod D), and will not be included in the nursing ratio until active. When the cases become active, the beds they are assigned to must become blocked in the configurator (e.g., the bed stats reporting system/configurator).

With reference again to FIG. 1, the optimization criteria data 132 can comprise information regarding the optimization objectives of the dynamic medical care facility system. In various embodiments, the optimization objectives can include but are not limited to: minimizing delays, maximizing patient flow, minimizing blocking, minimizing costs, and minimizing surgeon idle time.

In this regard, the objective of minimizing delays can include minimizing delays between workflow events (e.g., prepping for surgery, prepping for anesthesia, initiating anesthesia, movement from one perioperative phase of care to the next, etc.), minimizing patient wait times for case initiation (e.g., delays to start time), and other type of delays related to staff and system resource. In some implementations, the objective of minimizing delays can also include minimizing time delays between scheduled start times and actual start times (e.g., of cases and/or specific workflow events).

Maximizing patient flow refers to maximizing throughput; the frequency of completed cases over a defined time frame. For example, with respect to a perioperative system, maximining patient flow can include maximizing the number of patients moving through the system and receiving the surgery over a defined time frame, such as the entire workday, the peak hours (e.g., 8:00 am to 4:30 pm), or another defined time frame. In this regard, maximizing patient flow is related to minimizing delays because the act of minimizing delays can result in increasing patient flow. However, maximining patient flow is different from minimizing delays per se, because in some contexts, maximizing patient flow could add some delay to the system. For example, different surgical procedures can take different amounts of time to complete. If the objective is to maximize patient flow over peak hours (e.g., 8:00 am to 4:30 pm), one way to achieve this would be to perform the shorter procedural cases during this time frame to result in maximizing the number of cases performed over peak hours when for example, there are more resources (e.g., staff) to facilitate these procedures.

Patient blocking refers to preventing a patient from receiving a required element of care within a required or preferred time frame, wherein the required care elements and time frames can be defined in the by the corresponding case workflow (e.g., in the case workflow data 132b) and/or the system policy data 132d. In this regard, minimizing or preventing patient blocking is different from minimizing some delays, because in these cases, no amount of delay over a defined amount of time is acceptable, as it would result in preventing a patient from receiving the required care at the requisite time of need. For example, assuming a post-operative patient requires immediate transition to a bed with oxygen support and all beds with oxygen support are current occupied. This would be a case of blocking. Thus, one optimization objective can include minimizing and/or preventing the occurrence of these patient blocking scenarios.

Minimizing costs can include minimizing financial costs to the dynamic medical facility system that can be realized without compromising patient quality of care and access to care. For example, minimizing costs can be attributed to minimizing overtime pay, minimizing costs of attributed to complications, minimizing costs attributed to delays, minimizing costs attributed to underutilization or inefficient utilization of resources (e.g., staff, medical equipment/supplies, etc), minimizing costs attributed to wasteful utilization of resources (e.g., patient administered oxygen when not necessary).

Minimizing surgeon idle time refers minimizing surgeon wait time between procedures. In this regard, the surgeon's time is often the most valuable (i.e., costly to the system). Thus, delays in the perioperative system that result in the surgeon having idle time between procedures are attributed to higher losses.

In various implementations, the different optimization objectives are related and/or interconnected such that optimizing one can impact another in a positive or negative manner. In this regard, in some embodiments, the optimization criteria data 132e can also include weights regarding the relative importance of the respective objectives that can be used by the optimization component 118 in formulating an optimization solution that balances and/or prioritizes the objectives according to their weights. The weights can be predefined and/or user adjusted as appropriate to meet the systems objectives in different contexts (e.g., the weights can vary for different time-frames, different days of the week, different procedures, different surgeons, etc.).

In various embodiments, the medical facility system management module 104 facilitates determining how to optimally sequence, place and allocate resources for a dynamic medical facility system (e.g., a perioperative system) to achieve one or more of the optimization objectives based on the multitude of inputs included in the current state data 102, the historical state data 130 and the medical facility system data 132. In this regard, the reception component 106 can receive the current state data 102 from one or more current state data sources/system 101 associated with the medical care facility system in real-time over the course of operation of the dynamic medical facility. For example, the one or more current state data sources/systems 101 can include (but are not limited to): case scheduling systems, staff scheduling systems, case status tracking systems, patient location tracking systems, patient vital signs monitoring systems, resource status and location monitoring systems, bed status tracking systems, staff location tracking and activity monitoring systems, data entry systems and the like. The one or more current state data sources/systems 101 can also include data sources and/or systems associated with the entire perioperative system as a whole and/or associated with sub-specialty areas/modalities. In this regard, the reception component 106 can collect, extract or otherwise receive the current state data 102 continuously and/or regularly over the course of operation of the dynamic medical facility as the current state data 102 is generated by and/or entered into the one or more current state data systems/sources 101. For example, in some implementations, the reception component 106 can repeatedly extract, collect or otherwise receive sets of the active case data 102 reflective of a current state of the medical facility systems according to a defined schedule (e.g., every second, every few seconds, every minute, every five minutes, every ten minutes, etc.). In other implementations, the reception component 106 can receive (e.g., in a push fashion) active case data 102 reflecting any changes to the current state of the medical facility system as they occur (e.g., in response to entry into and/or generation by the one or more current state data sources/systems).

The forecasting component 108 can further employ a machine learning/artificial intelligence (AI) framework to regularly and/or continuously forecast future state information for the medical facility system in real-time based on the current state data 102, the historical state data 130, and/or relevant information included the medical facility system data 132 that can influence and/or control the timing of defined workflows for the patient cases (e.g., including timing of initiation, completion and duration of defined workflow events), and resources needed.

In one or more embodiments, the future state information can include timeline forecasts 136 regarding the future timing of initiation and/or completion of workflows and discrete workflow events of the active and pending patient cases. With these embodiments, forecasting component 108 can include case timeline forecasting component 110 to forecast the timeline information (e.g., the timeline forecasts) using one or more machine learning/AI techniques based on relevant factors included in the current state data 102, the historical state data 130, and/or relevant information included the medical facility system data 132 that can influence, correlate to, and/or control the timing of workflows for the patient cases (e.g., including timing of initiation, completion and duration of defined workflow events).

For example, the timeline forecasts 136 can include information for currently active cases that forecasts, based in part on multitude of parameters represented in the current state data 102 regarding the active cases and the current operating conditions of the medical facility system and relevant constraints defined by the medical facility system data 132, when the current workflow events for the active cases will be completed (e.g., surgery for patient John. B will be finish is 27 minutes or at 1:15 pm, patient Jane D. is expected to remain in the preoperative holding room for the next 55 minutes, etc.), and the duration of time between initiation and completion of downstream workflow events (e.g., following surgery, patient John. B is expected to be moved to bed in the PACU of type XYZ within 15 minutes or by 1:30 pm, following placement in the PACU, patient John. B is expected to remain in the PACU for 45 minutes and then be discharged at 2:15, and so one). According to this example, assuming an active patient case follows a defined workflow with sequential workflow events, based on case workflow data 132b defining the sequential events to be completed and associated rules/requirements for the workflow (e.g., regarding timing, placement, clinician requirements, etc.), the current status of the case, the type of the case, the current status of the patient, the current operating conditions of the medial facility system (e.g., bed availability, demand for beds based on other patients/case status), historical state data 130 regarding timing of same or similar case workflows for same or similar patients under same or similar operating conditions, historical efficiency of the specific clinicians assigned to the case with respect to performing certain workflow tasks (e.g., average time surgeon Jay Jones takes to perform this procedure), system policy rules regarding constraints placement periodization, staffing ratios, a variety of other relevant factors, the case timeline forecasting component 108 can regularly generate forecasted timeline information for the active case regarding when the current workflow event is expected to be completed, and timing of any remaining downstream workflow events (e.g., regarding start and end times of the downstream workflow events).

The case timeline forecasting component 110 can similarly forecast this same type of timeline information for pending cases. For example, with respect to pending cases, the case timeline forecasting component 110 can forecast when the pending case will begin (e.g., become “active” based on initiation of the first workflow event for the case), as well as future timeline information regarding the expected timing of initiation and completion of the workflow events for the pending case given then current state of the dynamic medical facility system and various other relevant historical factors, rules and constraints discussed herein (e.g., pending perioperative case No. 1918 is expected to begin preop at 3:35 pm, being surgery at 4:15 pm, finish surgery at 6:30 pm, be moved to ICU immediately following surgery, etc.). In this regard, the case timeline forecasting component 110 can employ learned correlations between timing of workflows (including timing of discrete workflow events) and a multitude of dynamic factors included in the current state data 102 and the medical facility system data 132 to forecast timelines identifying expected timing of events for active and pending case workflows.

In some embodiments, the future state information can also include resource demand forecasts 138 regarding forecasted demand for resources (e.g., staff, beds, equipment, supplies, etc.) at the medical facility system with respect to time (e.g., when the resources will be needed), amount (e.g., number of resources that will be needed), type (e.g., type of staff with respect to role/qualifications, type of medical supplies/equipment, etc.), and location (e.g., where the resources will be needed). With these embodiments, the forecasting component 108 can include resource demand forecasting component 114 to forecast the future resource demands using one or more machine learning/AI techniques based on the current state data 102, the historical state data 130, and/or relevant information included the medical facility system data 132 that can influence and/or control the amount, type, timing and location of resources needed by the dynamic medical facility system. For example, in implementations in which the dynamic medial facility system is a perioperative system, the resource demand forecasting component 114 can forecast what resource will be needed at the what areas of the perioperative system (e.g., specific floors, rooms, bays, pods, beds, etc.), at different times or time frames over a defined upcoming window of time (e.g., the next hour, the next 12 hours, the next 24 hours, the next 48 hours, etc.). For instance, the resource demand forecasting component 114 can forecast the number of nurses with “abc” qualifications needed in postoperative area A at 2:00 pm or from 2:00 pm to 3:00 pm, and so on.

In various embodiments, in addition to the various parameters included in the current state data 102, the historical state data 130 and the relevant rules, constraints and policies defined in the medial facility system data 132, the resource demand forecasting component 114 can use the timeline forecasts 136 as input to determine the forecasted resource demands. In this regard, based on forecasted information indicating when certain procedures and workflow events are expected to occur and what type/amount of resources are needed to perform or enable performance of the workflow events, the resource forecasting component 114 can forecast the future demand for the resources with respect to timing, amount, location, type, etc. For example, based on timeline forecasts 136 indicating 6 high acuity patients will be moved to PACU beds in Pod C between 5:00 and 6:00 pm and policy information requiring a nursing ratio of 1:2 (one nurse for two patients) for Pod C, the resource demand forecasting component 114 can determine/forecast that three nurses with qualifications of “xyz” will be needed in Pod C between 5:00 and 6:00 pm.

The case timeline forecasting component 110 and the resource demand forecasting component 114 can employ various machine-learning based techniques, statistical-based techniques and/or probabilistic-based techniques to generate the timeline forecasts 136 and the resource demand forecasts 138 based on the multitude of input parameters included in the current state data 102, the historical state data 130 the medical facility system data 132, (and the timeline forecasts 136 when used as input for determining the resource demand forecasts). The machine learning techniques can include supervised machine learning techniques, semi-supervised machine learning techniques, unsupervised machine learning techniques, or a combination thereof. For example, the case timeline forecasting component 110 and the resource demand forecasting component 114 can employ expert systems, fuzzy logic, SVMs, Hidden Markov Models (HMMs), greedy search algorithms, rule-based systems, Bayesian models (e.g., Bayesian networks), neural networks, other non-linear training techniques, data fusion, utility-based analytical systems, systems employing Bayesian models, etc. In another aspect, the case timeline forecasting component 110 and the resource demand forecasting component 114 can perform a set of machine learning computations associated with analysis of the current state data 102, the historical state data 130 the medical facility system data 132, (and the timeline forecasts 136). For example, the case timeline forecasting component 110 and the resource demand forecasting component 114 can perform a set of clustering machine learning computations, a set of logistic regression machine learning computations, a set of decision tree machine learning computations, a set of random forest machine learning computations, a set of regression tree machine learning computations, a set of least square machine learning computations, a set of instance-based machine learning computations, a set of regression machine learning computations, a set of support vector regression machine learning computations, a set of k-means machine learning computations, a set of spectral clustering machine learning computations, Gaussian mixture model machine learning computations, a set of regularization machine learning computations, a set of rule learning machine learning computations, a set of Bayesian machine learning computations, a set of deep Boltzmann machine computations, a set of deep belief network computations, a set of convolution neural network computations, a set of stacked auto-encoder computations and/or a set of different machine learning computations.

In some embodiments, the case timeline forecasting component 110 and the resource demand forecasting component 114 can perform learning with respect to the current state data 102, the historical state data 130, the medical facility system data 132, and the timeline forecasts 136 explicitly or implicitly. The case timeline forecasting component 110 and the resource demand forecasting component 114 can also employ an automatic classification system and/or an automatic classification process to facilitate analysis of the current state data 102, the historical state data 130, the medical facility system data 132 and the timeline forecasts 136. For example, the case timeline forecasting component 110 can employ a probabilistic and/or statistical-based analysis (e.g., factoring into the analysis utilities/rewards and costs) to learn and/or generate inferences regarding timing of case workflows with respect to the current state data 102, the historical state data 130, and the medical facility system data 132. Similarly, the resource demand forecasting component 114 can employ a probabilistic and/or statistical-based analysis (e.g., factoring into the analysis utilities/rewards and costs) to learn and/or generate inferences regarding resource needs (e.g., with respect to amount, type, timing and location) and the timeline forecasts 136, the current state data 102, the historical state data 130, and the medical facility system data 132. The case timeline forecasting component 110 and/or the resource demand forecasting component 114 can employ, for example, a support vector machine (SVM) classifier to learn and/or generate such inferences).

Additionally or alternatively, the case timeline forecasting component 110 and/or the resource demand forecasting component 114 can employ other classification techniques associated with Bayesian networks, decision trees and/or probabilistic classification models. Classifiers employed by the case timeline forecasting component 110 and/or the resource demand forecasting component 114 can be explicitly trained (e.g., via a generic training data) as well as implicitly trained (e.g., via receiving extrinsic information). For example, with respect to SVM's, SVM's can be configured via a learning or training phase within a classifier constructor and feature selection module. A classifier can be a function that maps an input attribute vector, x=(x1, x2, x3, x4, xn), to a confidence that the input belongs to a class—that is, f(x)=confidence(class).

In some embodiments, the case timeline forecasting component 110 can employ one or more timeline models 112 to forecast and/or facilitate forecasting the case timeline information (e.g., the timeline forecasts 136) based relevant parameters included in the current state data 102, the historical state data 130 and the medical facility systems data 132. The resource demand forecasting component 114 can similarly employ one or more resource demand models 116 to forecast and/or facilitate forecasting the resource demand forecasts 138 based on the timeline forecasts 136 and relevant parameters included in the current state data 102, the historical state data 130 and/or the medical facility systems data 132. In this regard, the one or more timeline models 112 and/or the one or more resource demand models 116 can include a variety of machine learning model types, including but not limited to: a nearest neighbor model, naïve Bayes model, a decision tree model, a boosting model, a linear regression model, a neural network model, a deep neural network model, a k-means clustering model, an association rules model, q-learning model, a temporal difference algorithm, a deep adversarial network model, a classification model, or a combination thereof.

In one example, the one or more timeline models 112 can include a classification machine learning model that maps relevant parameters included in the current state data 102, the historical state data 130 and/or the medical facility system data 132 to one or more categories. In another example, the one or more timeline models 112 can include a regression machine learning model or neural network model that determines relationships amongst relevant parameters included in the current state data 102, the historical state data 130 and/or the medical facility system data 132 and timing of specific workflows and workflow events. In yet another example, the one or more timeline models 112 can include clustering machine learning model that groups related data from the current state data 102, the historical state data 130 and/or the medical facility system data 132 into a corresponding group.

Similarly, the one or more resource demand models 116 can include a classification machine learning model that maps relevant parameters included in the timeline forecasts 136, the current state data 102, the historical state data 130 and/or the medical facility system data 132 to one or more categories. In another example, the one or more resource demand models 116 can include a regression machine learning model or neural network model that determines relationships amongst relevant parameters included in the timeline forecasts 136, the current state data 102, the historical state data 130 and/or the medical facility system data 132 and resource needs with respect to amount, type (e.g., staff with specific qualifications, specific medical supplies, etc.), timing and location. In yet another example, the one or more resource demand models 116 can include a clustering machine learning model that groups related data from the timeline forecasts 136, the current state data 102, the historical state data 130 and/or the medical facility system data 132 into a corresponding group.

In some embodiments, the case timeline forecasting component 110 and/or the resource demand forecasting component 114 can employ one or more artificial intelligence techniques to execute the timeline models 112 and/or the resource demand models 116 respectively, based on the current state data 102, the historical state data 130, the medical facility system data 132, and the timeline forecasts 136. For example, the case timeline forecasting component 110 can extract relevant information from the current state data 102, the historical state data 130 and the medical facility system 132 data that is indicative of correlations, inferences and/or expressions associated with timing of case workflows based on principles of artificial intelligence. The case timeline forecasting component 110 can further generate the timeline forecasts 136 based on the execution of the one or more timeline models 112 using the relevant extracted information. Similarly, the resource demand forecasting component 114 can extract relevant information from the timeline forecasts 136, the current state data 102, the historical state data 130 and the medical facility system data 132 that is indicative of correlations, inferences and/or expressions associated with resource need (e.g., with respect to amount, type, timing and location) based on principles of artificial intelligence. The resource demand forecasting component 114 can further generate the resource demand forecasts based on the execution of the one or more resource demand models 116 using the relevant extracted information.

In various embodiments, the one or more timeline models 112 and/or the one or more resource demand models 116 can include previously trained/developed (e.g., developed in accordance with existing machine learning/development techniques using training, test and validation sets). For example, the one or more one or more timeline models 112 can include one or more models trained to predict case workflow timeline information based on learned correlations between timing of case workflows (including overall timing and timing of discrete workflow events) and various relevant parameters (and/or the parameter values) included in the historical state data 130 and the medical facility system data 132. With these embodiments, the case timeline forecasting component 110 can thus identify and extract these relevant parameters (and/or the parameter values) from the current state data 102 and the medical facility system data 132 at runtime for input to the one or more timeline models 112 to generate the timeline forecasts 136. Similarly, the one or more one or more resource demand models can include one or more models trained to predict resource demands (e.g., with respect to amount, type, timing and location) based on learned correlations between resource needs and various relevant parameters (and/or the parameter values) included in the historical state data 130 and the medical facility system data 132. With these embodiments, the resource demand forecasting component 114 can thus identify and extract these relevant parameters (and/or the parameter values) from the current state data 102, the medical facility system data 132, and the timeline forecasts 136 at runtime for input to the one or more resource demand models 116 to generate the resource demand forecasts 138. It should be appreciated that the specific input features extracted from the current state data 102 and the medical facility system data 132 and fed into the timeline models 112 and the resource demand models 116 can vary (e.g., the respective models can be configured to process different input sets). With these embodiments, the timeline models 112 and the resource demand models 116 can further be regularly updated over time based on new historical data 130.

The optimization component 118 can further employ a complex heuristic-based optimization mechanism to determine optimal reactive solutions regarding patient sequencing, patient placement and/or resource allocation that achieves and/or balances (e.g., in accordance with defined weights) the one or more optimization objectives (e.g., as provided by the optimization criteria data 132e) based on relevant parameters included in the current state data 102, the future state information (e.g., including the timeline forecasts 136 and/or the resource demand forecasts 138), and defined system constraints (e.g., system architecture constraints, defined workflow constraints, defined staffing constraints, and defined system rule/policy based constraints, as respectively provided by the medical facility system data 132) that control or influence patient sequencing and timing, placement and/or resource allocation. In the embodiment shown, optimal solutions regarding patient sequence and timing are identified as patient sequence and timing solutions 140, optimal solutions regarding patient placement are identified as patient placement solutions 142, and optimal solutions regarding resource allocation are identified as resource allocation solutions 144. In this regard, the optimization component 118 can perform a complex optimization routine continuously as patients arrive, schedules change, staff move between areas and add-ons/cancelations occur to determine how to sequence and place patients in real-time using a complex heuristic-based algorithm with embedded rules and machine learning components (e.g., corresponding to the timeline forecasts 136 and the resource demand forecasts 138) to optimize the sequencing and placement of patients in an environment of shared and sub-specialized resources.

For example, with respect to a perioperative system, the optimization component 118 can model the entire perioperative system as a large-scale combinatorial optimization problem with fixed constraints that control or influence patient sequencing and timing, placement and/or resource allocation (e.g., as defined be medical facility system data 132) and one or more optimization objectives, and adjustable variables corresponding to patient/case sequencing and timing, patient placement (e.g., with respect to a physical location/area), and resource allocation (e.g., assignment of staff and other resources). The optimization component 118 can further iteratively adjust these variables based on the current state data 102, the timeline forecasts 136 and the resource demand forecasts 138 to converge on optimal solutions regarding patient sequencing and timing, patient placement and/or resource allocation that “best” achieves and/or balances (e.g., in accordance with defined weights) the one or more optimization objectives. The optimization component 118 can further continuously and/or regularly (e.g., every minute, every 15 minutes, every 30 minutes, every hour, every 6 hours, every 12 hours, etc.), perform this optimization process to generate real-time solutions that reflect the current state of the system.

In the embodiment shown, the optimization component 118 can include a sequence and timing optimizer 120, a patient placement optimizer 122 and a resource allocation optimizer 124. In various embodiments, the sequence and timing optimizer 120 can be configured to determine or facilitate determining optimal patient sequence and timing solutions 140, the patient placement optimizer 122 can be configured to determine or facilitate determining the optimal patient placement solutions 142, and the resource allocation optimizer 124 can be configured to determine optimal resource allocation solutions 144.

For example, the sequence and timing optimizer 120 can determine how to order or sequence initiating performance of pending patient cases based on relevant parameters included in the current state data 102, the timeline forecasts 136, the resource demand forecasts 138, and relevant constraints included in the medical facility system data 132, that best achieves and/or balances the one or more optimization objectives. In this regard, the sequence and timing optimizer 120 can adjust patient scheduling in real-time (e.g., by reordering the start times of the respective pending cases) based on relevant parameters included in the current state data 102, the timeline forecasts 136, the resource demand forecasts 138, and relevant constraints included in the medical facility system data 132, to converge on a new sequence for performing the pending cases that minimizes delays (e.g., over a defined timeframe), maximizes patient flow (e.g., over a defined timeframe), minimizes or eliminates blocking (e.g., over a defined timeframe), minimizes costs (e.g., over a defined timeframe), and/or minimizes surgeon idle time. If more than one optimization objective is integrated into the optimization problem, the sequence and timing optimizer 120 can employ a defined weighting scheme for balancing the respective objectives.

Importantly, the sequence and timing optimizer 120 can employ a time-based heuristic to determine sequence and timing information that accounts for accurate timeline forecasts 136 regarding when patients, staff and resources will be ready and available to begin a next workflow or workflow event. For instance, to provide a basic example considering only a piece of the timeline forecasts 136, assume a perioperative environment that performs various surgical procedures in operating room A, operating room B, and operating room C. Based on timeline information indicating the procedure currently being performed in operating room A will finish first, the surgery in operating room B will finish second, and the surgery in operating room C will finish third, the sequence and timing optimizer 120 can determine an optimal order for initiating the pending patient cases that would minimize system delays and maximize throughput would include first initiating a pending case that requires surgery in operating room A, then initiating a pending patient case that requires surgery in operating room B, and lastly initiating a pending patient case for a patient that requires surgery in operating room C.

In some implementations, in addition to determining how to order or reorder pending patient cases, the sequence and timing optimizer 120 can also determine actual start times for initiating the pending cases that best achieves and/or balances the one or more optimization objectives based on the current state data, the timeline forecasts 138, the resource demand forecasts 138 and the system constraints. Thus, in various embodiments, the patient sequence and timing solutions 140 can provide a recommended order for initiating pending cases and in some implementations, information identifying recommended start times for initiating the respective pending cases.

The sequence and timing optimizer 120 can also determine how to sequence and time performance of specific workflow events for active and pending cases. For example, the sequence and timing optimizer 120 can determine an order and/or timing for initiating specific workflow events for different cases based on relevant parameters included in the current state data 102, the timeline forecasts 136, the resource demand forecasts 138, and relevant constraints included in the medical facility system data 132, that best achieves and/or balances the one or more optimization objectives. For instance, assuming several active cases require administration of anesthesia within the next hour, the sequence and timing optimizer 120 can determine how to order administration of the anesthesia to the respective patients in a manner that results in best minimizing delays, prevents downstream blocking, minimizes surgeon wait times, etc. In this regard, the recommended order can be asynchronous with respect to the timing of arrival of the respective cases in the pre-operative area for surgery preparation. The sequence and timing optimizer 120 can also determine timing for initiating specific workflow events for individual cases based on relevant parameters included in the current state data 102, the timeline forecasts 136, the resource demand forecasts 138, and relevant constraints included in the medical facility system data 132, that best achieves and/or balances the one or more optimization objectives. For example, the sequence and timing optimizer 120 can determine that an active case for a patient currently in PACU should move the patient to a post-operative recovery room at 3:55 to open up the PACU bed for a patient requiring the bed at 4:10 (e.g., when a minimum of 15 minutes is required for bed cleaning). Thus, in various embodiments, the patient sequence and timing solutions 140 can provide a recommended order and/or timing for initiating specific workflow events.

The patient placement optimizer 122 can determine where to place patients at different procedural areas throughout their workflows based on relevant parameters included in the current state data 102, the timeline forecasts 136, the resource demand forecasts 138, and relevant constraints included in the medical facility system data 132, that best achieves and/or balances the one or more optimization objectives. For example, assume a perioperative system in which patients are moved to different procedural areas (e.g., different units, rooms, pods, bays, beds, etc.) with different resources and/or specialty care services in association with different phases of the preoperative process. In accordance with this example, assuming the preoperative system has limited resources (e.g., limited beds and staff), the patient placement optimizer 122 can determine which patients to place in specific preoperative, interoperative and postoperative areas/beds at different times during their preoperative process that accounts for the current and future workflow timing of all cases, considers all patients different medical needs at different times (e.g., different levels of acuity and priority for different types of beds), considers placement prioritization constraints in view of bed availability (e.g., higher priority/acuity patients get placed in higher service level beds), considers staffing constraints in view of current and future staffing assignments/availability (e.g., regarding nursing ratios, qualifications to treat certain patients based on their levels of acuity and care needs), and the like, in a manner that best achieves or balances the one or more optimization objectives (e.g., minimizes delays, maximizes patient flow, minimizes or prevents blocking, minimizes costs, and minimizes surgeon wait times). In this regard, the patient placement solutions 142 can include information that assigns patients/cases to specific procedural areas/beds as required by their workflows and care needs over their course of care.

The resource allocation optimizer 124 can similarly determine how to allocate system resources based on relevant parameters included in the current state data 102, the timeline forecasts 136, the resource demand forecasts 138, and relevant constraints included in the medical facility system data 132, that best achieves and/or balances the one or more optimization objectives. For example, in some embodiments the resource allocation optimizer 124 can evaluate the resource demand forecasts 138 providing information identifying the expected amount, type, location of resources needed at defined upcoming times or time frames and allocated resource accordingly to meet the expected demand. For example, based on forecasted resource demand information indicating that 3 nurses are needed in unit A from 1:00 to 2:00 pm, 5 nurses are needed from 2:00 to 3:00 pm, 4 nurses are needed from 3:00 to 4:00 pm, etc., the resource allocation optimizer can identify available nurses in other units and re-assign them to unit A accordingly. In addition to allocating resources to meet the expected demands, the resource allocation optimizer can also consider many other variables including constraints regarding staff availability, qualifications/credentials and patient needs, required nursing ratios, costs attributed to overtime pay, demand in other units, prioritization schemes for patients and resources, and the like in view of the one or more optimization objectives. For example, the resource allocation optimizer can further determine how to assign staff to patients/cases and physical areas of the perioperative system (e.g., units, rooms, floors, pods, bays, beds, etc.) to minimize delays, maximize patient flow, prevent blocking, minimize costs, minimize surgeon wait times, etc., while balancing staff workload (e.g., toward on equal distribution of the workload amongst the available staff) in view of staff availability, qualifications, and patient needs. The resource allocation optimizer can also apply constraints regarding assignment restrictions, shift constraints (e.g., timing of shifts, maximum and minimum job allocation per staff member per shift, etc.) and capacity constraints (e.g., regarding system capacity) in association with task assignment rules (e.g., fair distribution of task rules, policy, zone rules, patient and material transport rules, etc.) to determine how to optimize the assignment of staff members to the tasks (e.g. to optimize the number of tasks fulfilled and balance the distribution of the workload). In this regard, the resource allocation optimizer 124 can determine resource allocation solutions 144 that staff and other resources to patients (or vice versa) that best meets system demands while balancing the optimization objectives.

In many implementations, optimal solutions regarding patient sequence and timing, patient placement and resource allocation are highly interrelated. In this regard, in addition to evaluating these solutions in isolation, the optimization component 118 can further employ combinatorial optimization techniques to iteratively evaluate different combinations of patient sequence and timing solution options determined by the sequence and timing optimizer 120, patient placement solution options determined by the patient placement optimizer, and resource allocation solution options determined by the resource allocation optimizer 124, to converge on an optimal combination of these solutions that best achieves and/or balances (e.g., in accordance with a defined weighting schemed) the one or more optimization objectives.

The reporting component 126 can further provide output data 134 including the future state information generated by the forecasting component 108 and the solutions information regarding the optimal reactive solutions determined by the optimization component 118 as they are respectively forecasted and determined in real-time to one or more entities at the medical care facility system to facilitate managing operations of the medical care facility in real-time. For example, in the embodiment shown, the reporting component 126 can generate output data 134 that can the include the timeline forecasts 136, the resource demand forecasts 138, the patient sequence and timing solutions 140, the patient placement solutions 142 and the resource allocation solutions 144. In various embodiments, the output data 134 can be provided to relevant entities or users (e.g., staff at the medical facility) in real-time via a graphical user interface (GUI) tile at one or more devices associated with the relevant entities or users. The output data 134 can be reported using various suitable data structures (e.g., as machine readable text, as human readable text, as a graphical visualization, etc.) and/or electronic rendering applications. For example, in some embodiments, the reporting component 126 (and/or one or more components of the medical facility system management module 104) can be integrated with or be coupled to one or more applications that provide various features and/or functionalities associated with managing a dynamic medical facility system (e.g., a perioperative system). For instance, in one implementation, the application can include care delivery management application accessible via a network-based platform (e.g., a web-application, a website, a thin client application), a centralized command center, or another suitable operating environment. The display component 128 can further provide one or more tiles reporting the output data 134 in real-time.

For example, in the embodiment shown, the reporting component 126 can include a display component 128 configured to facilitate generating a graphical visualization and/or graphical user interface (GUI) for displaying at one or more user devices (not shown) that includes the timeline forecasts 136, the resource demand forecasts 138, the patient sequence and timing solutions 140, the patient placement solutions 142, and/or the resource allocation solutions 144. The one or more user devices can include for example, display monitors and/or computing devices associated with entities involved in the care delivery process at the dynamic medical facility system and/or involved in the patients care (e.g., staff of the perioperative system, the patients themselves, friends/family of the patients, etc.). In this regard, the visualization can allow a care provider, a perioperative system manager, a patient, a family member responsible for the patient, or the like, to view and receive updated information regarding the progress of the patient's workflow and expected timing of initiation and completion of workflow events including completion of the workflow (e.g., timeline forecasts), forecasted need for resources, recommended or applied patient sequence and timing solutions, recommended or applied patient placement solutions, and/or recommended or applied resource allocation assignments.

In this regard, the deployment architecture of system 100 and other systems described herein can vary. For example, various features and functionalities of system 100 (and additional systems described herein) can be deployed using a distributed computing environment, wherein the one or more devices, sources, modules and/or components of system 100 (and other systems described herein) can be provided in a distributed manner across various interconnected (via one or more networks) systems and devices (e.g., internal systems, the cloud, two or more dedicated servers, etc.). For example, system 100 can be deployed in a cloud architecture, a virtualized enterprise architecture, or an enterprise architecture wherein one the front-end components and the back-end components are distributed in a client/server relationship. With these embodiments, one or more features and functionalities of the system 100 can be deployed as a web-application, a cloud-application, a thin client application, a thick client application, a native client application, a hybrid client application, or the like, wherein one or more of the front-end components (e.g., reporting component 126) are provided at client device (not shown) and one or more of the back-end components (e.g., the data collection component 112, care outcomes forecasting component 114, etc.) are provided in the cloud, on a virtualized server, a virtualized data store, a remote server, a remote data store, a local data center, etc. (not shown), and accessed via a network (e.g., the Internet). In this regard, the current state data systems/sources 102, the medical facility system management module 104, one or more components of the medical facility management module, the historical state data 132 and/or the medical facility system data 132 can be physically separated yet communicatively coupled via one or more networks. However, it should be appreciated however that system 100 is not limited to this architectural configuration. For example, in another embodiment, the entirety of the medical facility system management module 104 can be deployed on a single local device (e.g., a desktop computer) as a local web-based application.

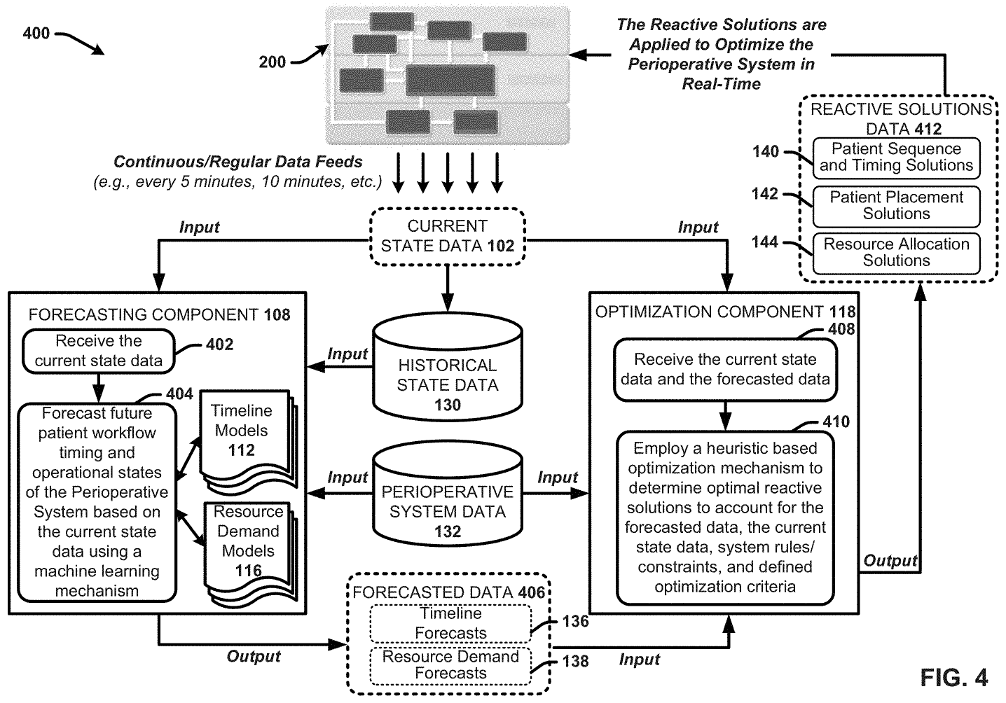

Turning now to FIG. 4, presented is a high-level flow diagram an example, non-limiting process 400 for optimizing the sequencing and placement of patients in a dynamic medical system using a continuous predictive-reactive scheme in accordance with one or more embodiments of the disclosed subject matter. Repetitive description of like elements employed in respective embodiments is omitted for sake of brevity.

In various embodiments, the forecasting component 108 and the optimization component 118 can perform a continuous predictive/reactive process to facilitate managing the operations of a dynamic medical facility (e.g., a perioperative system 200) in real-time over a course of operation of the dynamic medical facility system. In this regard, the continuous predictive/reactive process can involve regularly or continuously determining forecasted data 406 (e.g., including the timeline forecasts 136 and the resource demand forecasts 138) for the dynamic medial facility system by the forecasting component 108. For example, in accordance with process 400, at 402, the forecasting component 108 can receive current state data 102 for the perioperative system as continuous or regular data feeds (e.g., every 5 minutes, every 10 minutes, etc.). At 404, the forecasting component 108 can forecast future patient workflow timing and operational states of the perioperative system 200 based on the current state data using a machine learning mechanism. For example, the forecasting component 108 can employ relevant parameters included in the current state data 102, the historical state data 130, and/or the perioperative system data 132, as input to one or more timeline models 112, and one or more resource demand models 116 to generate the timeline forecasts 136 and/or the resource demand forecasts 138.

The optimization component 118 can further regularly or continuously employ the forecasted data 406 as input one or more optimization heuristics to regularly or continuously determine reactive solutions data 412 for the dynamic medical facility system (e.g., including the patient sequence and timing solutions 140, the patient placement solutions 142 and/or the resource allocation solutions 144). For example, at 408, the optimization component can receive the current stat data and the forecasted data 406. At 410, the optimization component 118 can employ a heuristic based optimization mechanism to determine optimal reactive solutions to account for the forecasted data 406, and relevant parameters included in the current state data 102, and the perioperative system data (e.g., system rules/constraints and defined optimization criteria).

The determined reactive solutions data 412 can further be automatically applied and/or applied with manual discretion in real-time to control or guide the operations of the dynamic medical facility system. As a result, the state of the dynamic medical facility system can change (e.g., based on implementing the recommended patient sequence and timing solutions, the patient placement solutions 142 and/or the resource allocation solutions 144), which will further be reflected in the newly generated current state data 102. The forecasting component 108 can further account for the system response when again evaluating the current state data 102 to determine updated forecasted data, and the optimization component 406 can again account for the updated forecasted data when determining new reactive solutions data. This predictive/reactive process can be iteratively performed over a course of operation of the dynamic medical facility system (e.g., every 5 minutes, every 10 minutes, etc.) to provide real-time solutions that account for the current state of the dynamic medical facility system.

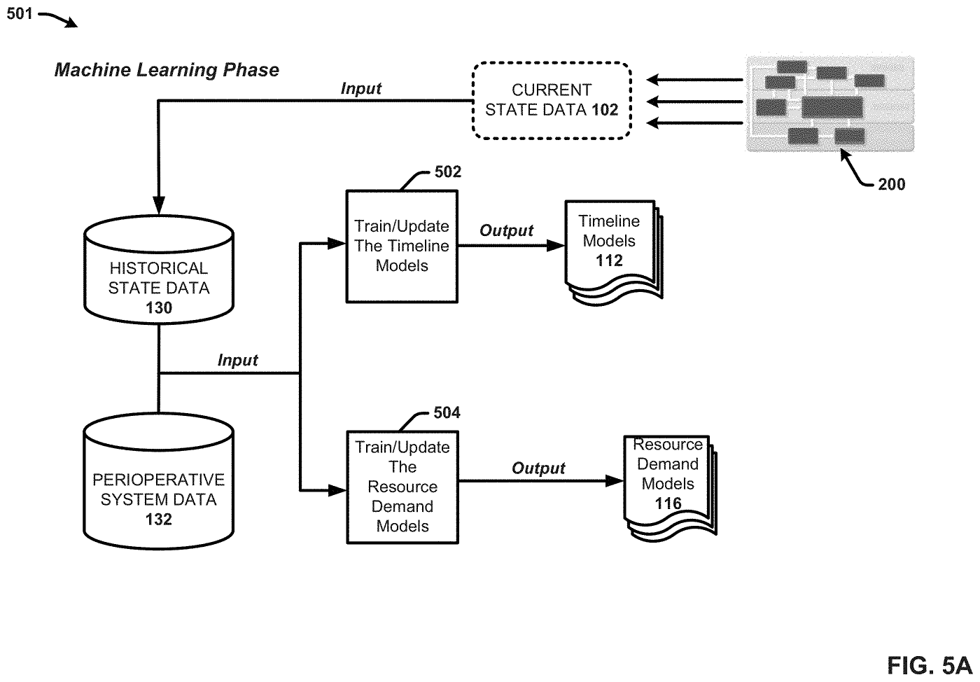

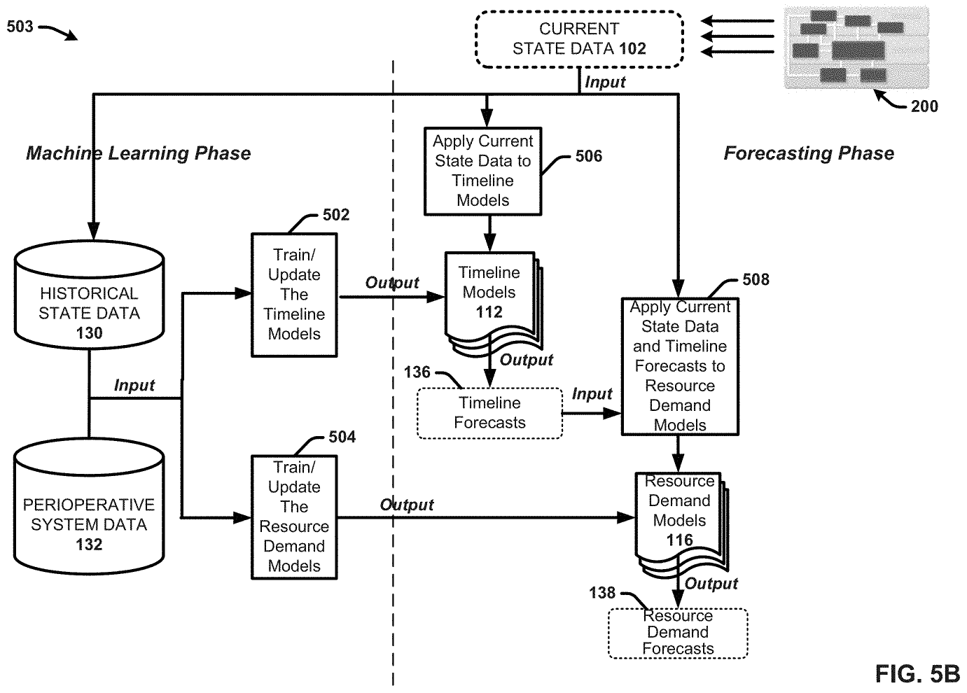

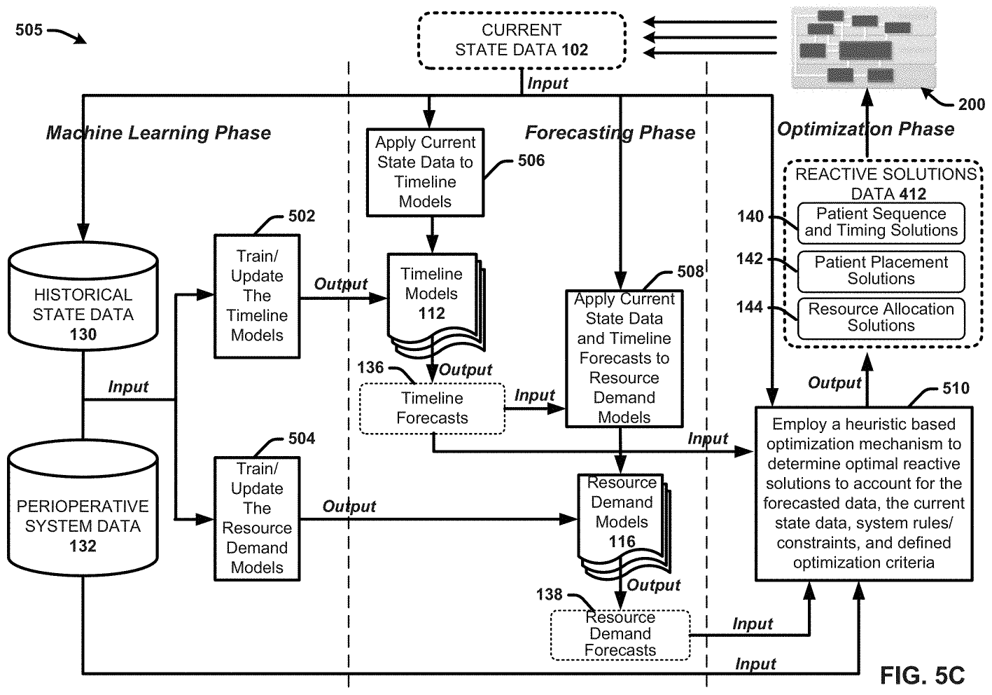

FIGS. 5A-5C respectively present flow diagrams illustrating different interrelated phases of a predictive/reactive process for managing the operations of a dynamic medical facility (e.g., a perioperative system 200) in real-time over a course of operation of the dynamic medical facility system in accordance with one or more embodiments described herein. These interrelated phases respectively include a machine learning phase, a forecasting phase, and an optimization phase. Repetitive description of like elements employed in respective embodiment is omitted for sake of brevity.

With reference initially to FIG. 5A, illustrated is a high-level flow diagram an example, non-limiting process 501 for training and updating machine learning models configured to forecast future states of a dynamic medical system based on a current state of the dynamic medical system in accordance with one or more embodiments of the disclosed subject matter. In this regard, process 501 demonstrates the machine learning phase. In accordance with process 501, current state data 102 for a dynamic medical facility system (e.g., a perioperative system 200) can be collected and aggregated as historical state data 130 over time. At 502, the historical state data 130 and the perioperative system data 132 can be used to train and/or update the one or more timeline models 112 (e.g., by the case timeline forecasting component 110). At 504, the historical state data 130 and the perioperative system data 132 can also be used to train and/or update the one or more resource demand models 504 (e.g., by the resource demand forecasting component 114).

FIG. 5B illustrates a high-level flow diagram an example, non-limiting process 503 for forecasting futures states of a dynamic medical system based on a current state of the dynamic medical system in accordance with one or more embodiments of the disclosed subject matter. In this regard, process 503 demonstrates addition of the forecasting phase in accordance with one or more embodiments. In accordance with process 503, once the timeline models 112 have been ben trained and/or updated, at 506, the case timeline forecasting component 110 can apply the current state data to the timeline models to generate the timeline forecasts 136. In addition, once the one or more resource demand models 116 have been trained and/or updated and the timeline forecasts 136 have been generated, at 508, the resource demand forecasting component 114 can apply the current state data 102 and the timeline forecasts to the resource demand models 116 to generate the resource demand forecasts 138.

FIG. 5C illustrates a high-level flow diagram another example, non-limiting process 505 for optimizing the sequencing and placement of patients in a dynamic medical system using a continuous predictive-reactive scheme in accordance with one or more embodiments of the disclosed subject matter. In this regard, process 505 demonstrates addition of the optimization phase in accordance with one or more embodiments. In accordance with process 505, once the timeline forecasts 136 and the resource demand forecasts 138 have been generated, at 510 the optimization component 118 can employ a heuristic based optimization mechanism to determine optimal reactive solutions (e.g., reactive solutions data 412) to account for the forecasted data (e.g., the timeline forecasts 136 and the resource demand forecasts 138), relevant parameters included the current state data 102, and the relevant system rules/constraints and the defined optimization criteria provided by the perioperative system data 132.

The determined reactive solutions data 412 can further be automatically applied and/or applied with manual discretion in real-time to control or guide the operations of the perioperative system 200 in real-time. As a result, the state of the perioperative system 200 can change (e.g., based on implementing the recommended patient sequence and timing solutions, the patient placement solutions 142 and/or the resource allocation solutions 144), which will further be reflected in the newly generated current state data 102. The machine learning phase can further be regularly performed as new current state data 102 is added to the historical state data 130 over time to update and/or optimize the timeline models and the resource demand models. The predictive/reactive processes of the forecasting phase and the optimization phase can also be iteratively performed over a course of operation of the dynamic medical facility system (e.g., every 5 minutes, every 10 minutes, etc.) to provide real-time solutions that account for the current state of the dynamic medical facility system.

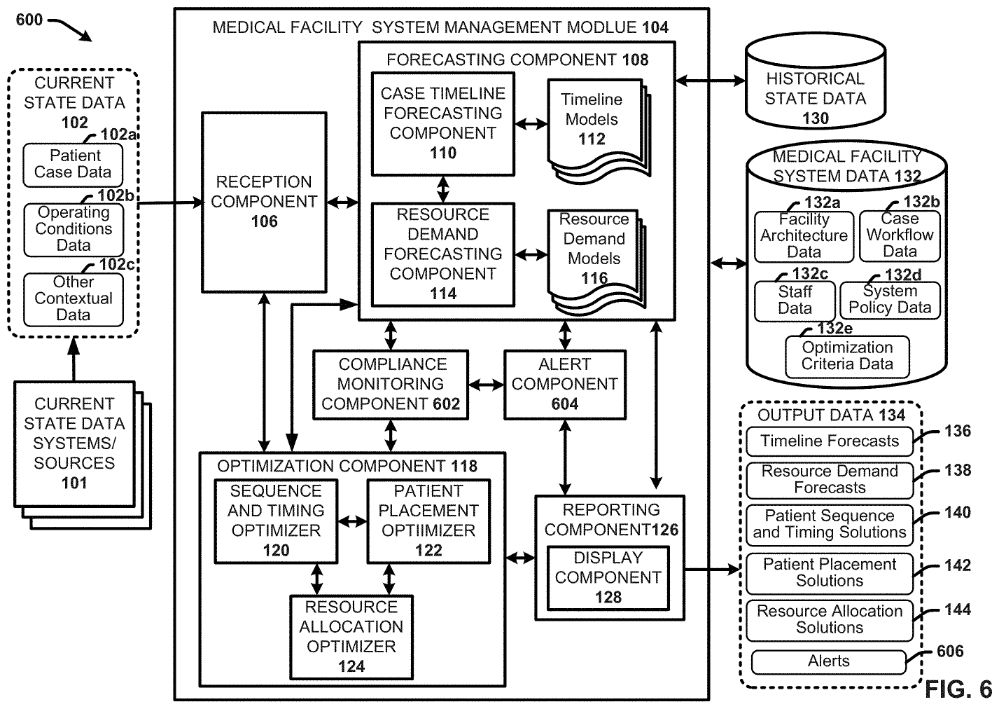

FIG. 6 illustrates a block diagram of another example, non-limiting system 600 for optimizing the sequencing and placement of patients in a dynamic medical system with shared and sub-specialized resources in accordance with one or more embodiments of the disclosed subject matter. System 600 include same or similar features and functionalities as system 100 with the addition of compliance monitoring component 602, alert component 604 and alerts 606. Repetitive description of like elements employed in respective embodiments is omitted for sake of brevity.

In various embodiments, the compliance monitoring component 602 can monitor and evaluate the timeline forecasts 136 and the resource demand forecasts 138 in view of the possible reactive solutions (e.g., the optimal patient sequence and timing solutions 140, the patient placement solutions 142 and/or the resource demand solutions 144) to determine whether the forecasted case workflow timing and/or the forecasted demand needs violate any defined rules/policies of the dynamic medical facility system (e.g., as defined in the medical facility system data 132). For example, the compliance monitoring component 602 can examine the timeline forecasts in view of the recommended patient sequence and timing solutions 140, the recommended patient placement solutions 142, and/or the resource allocation solutions 144, to determine whether a required optimization objective cannot be met. For example, the compliance monitoring component 602 can determine whether a maximum amount of delay allowed will be exceeded, whether a minimum patient flow rate for a particular timeframe cannot be met, whether a potential blocking scenario is identified, whether an amount of surgeon idle time will exceed the maximum allowed amount and the like. The alert component 604 can further generate alerts 606 regarding any identified or potential violation.

Similarly, the compliance monitoring component 602 can evaluated the forecasted demand in view of current and scheduled resource assignments (e.g., staff assignments) to determine whether available system resources at the different procedural areas at the different times comply with a resource constraint for the perioperative system based on forecasted demand. For example, the compliance monitoring component 602 can determine whether required nursing ratio will not be met in certain areas based on the forecasted demand (e.g., number of patients being placed in an area) and information regarding a known amount of nurses that are current assigned to and/or will be assigned to that procedural area. In this regard, the compliance monitoring component 602 can identify imbalances between the forecasted demand for the resources and available system resources at the different procedural areas at the different times. The alert component 604 can similarly generate alerts regarding identified resource imbalances. In addition, the optimization component 118 can further employ the heuristic-based optimization mechanism to determine the optimal reactive solutions based on the identified imbalances.

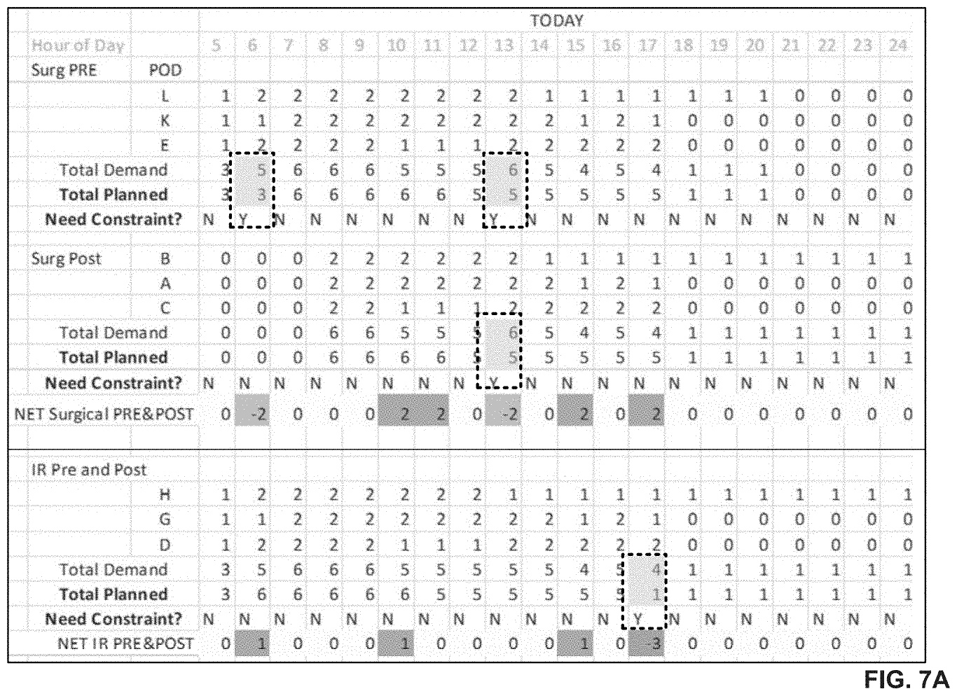

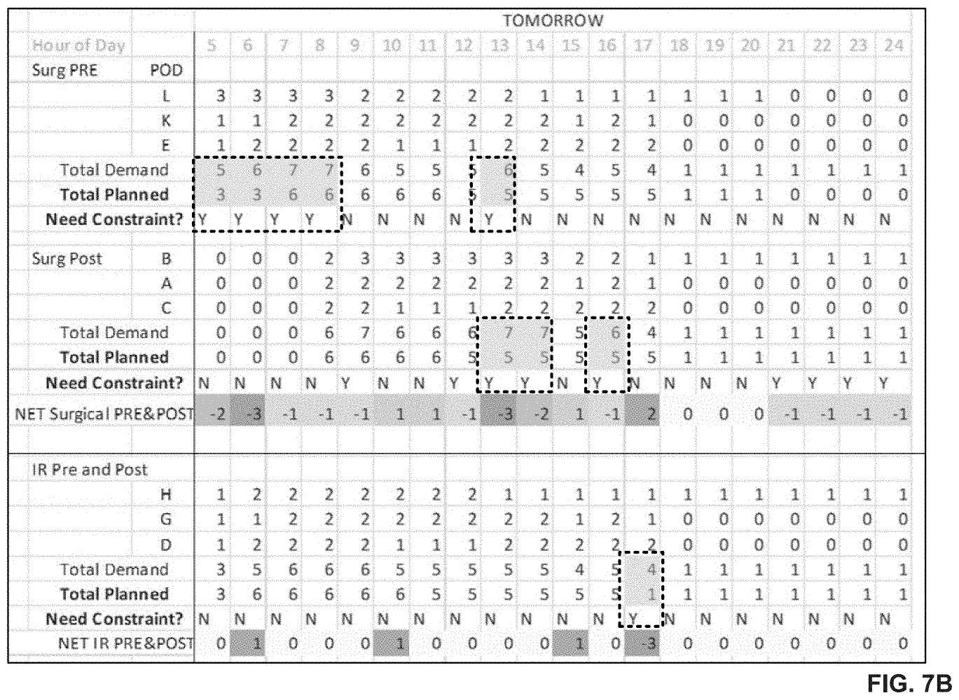

For example, FIGS. 7A and 7B provide a chart that identifies forecasted resource demands and identified resource supply/demand imbalances for a perioperative system over an upcoming timeframe in accordance with one or more embodiments of the disclosed subject matter. In the embodiment shown in FIGS. 7A and 7B, the chart encompasses the current workday (i.e., “TODAY”), as shown in FIG. 7A, and the following workday (i.e., “TOMORROW”), as shown in FIG. 7B. The chart provides information for each hour of the workday regarding forecasted resource demands for nurses in different Pods in the general surgery department as divided in to preoperative Pods (e.g., Pods L, K and E), and postoperative Pods (e.g., B, A and C). The chart also provides similar informaiton for the combined preoperative and postoperative Pods associated with the IR department (e.g., Pods H, G and D). For the respective hours of the day, the chart identifies the total expected demand of nurses needed (e.g., as “Total Demand”) for each group of Pods, either the first group of Pods corresponding to the preoperative surgery Pods (e.g., Pods L, K and E), the second group of Pods corresponding to the postoperative surgery Pods (e.g., Pods B, A and C), or the third group of Pods corresponding to the pre and post IR Pods (e.g., Pods H, G, and D). For the respective hours of the day, the chart also identifies the total amount of nurses planned/scheduled for assignment to the respective Pod groups. The dashed boxes are drawn around identified imbalances, where the total forecasted demand exceeds the total planned demand. Accordingly, a constraint is triggered (as marked with “Y” to indicate a constraint is need) for processing by the optimization component 118 to determine how to remedy the imbalance. In addition, the compliance monitoring component 602 can also identify situation in which the Net amount of planned/scheduled resources exceeds the forecasted demand. The optimization component 118 can also use this informaiton to determine where to pull resources from to redistribute as appropriate to account for the identified imbalances.

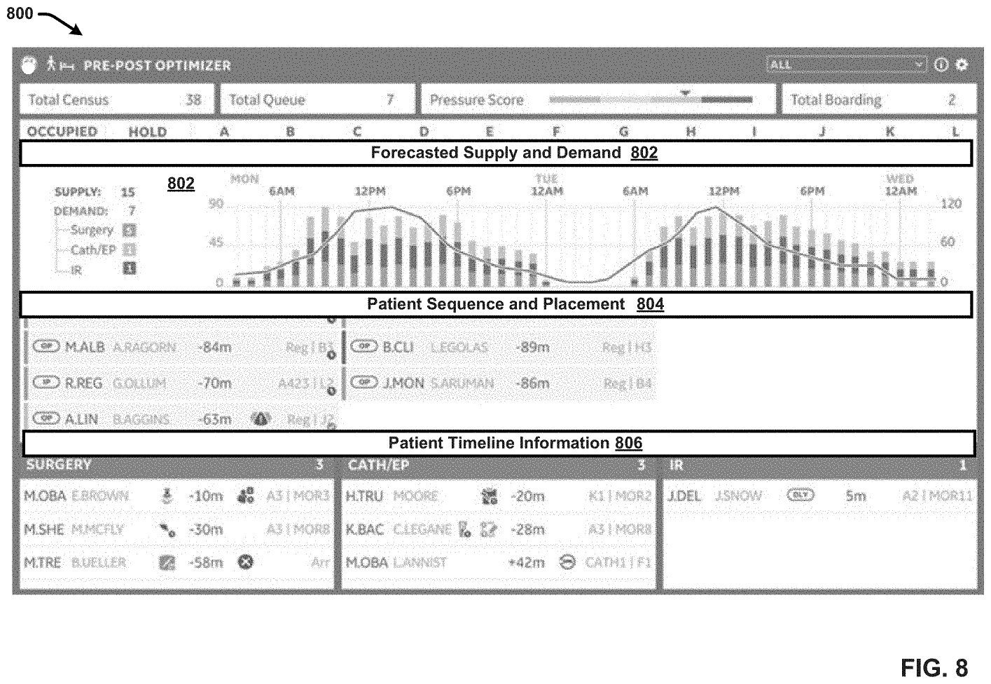

FIG. 8 presents an example dashboard 800 for presenting to clinicians comprising real-time information regarding the current and forecasted state of a dynamic medical system and optimized patient placement and sequencing information in accordance with one or more embodiments of the disclosed subject matter. In various embodiments, dashboard 800 presents an example UI in the form of a command center tile (e.g., an application that can be generated and/or presented by the display component 128. The dashboard 800 can present a variety of relevant informaiton regarding the timeline forecasts 136, the resource demand forecasts 138, the patient sequence and timing solutions 140, the patient placement solutions, 142, the resource allocation solutions 144 and/or the alerts 606. For example, in the embodiment shown, section 802 presents forecasted supply and demand information. Section 804 presents a specific sequence and placement of patients determined to minimize delay, maximize efficiency and satisfy a variety of rules and constraints. Alerts are shown in symbols regarding supply/demand imbalances or other violations. Section 806 presents patient timeline information for active patient cases in different surgical modalities.

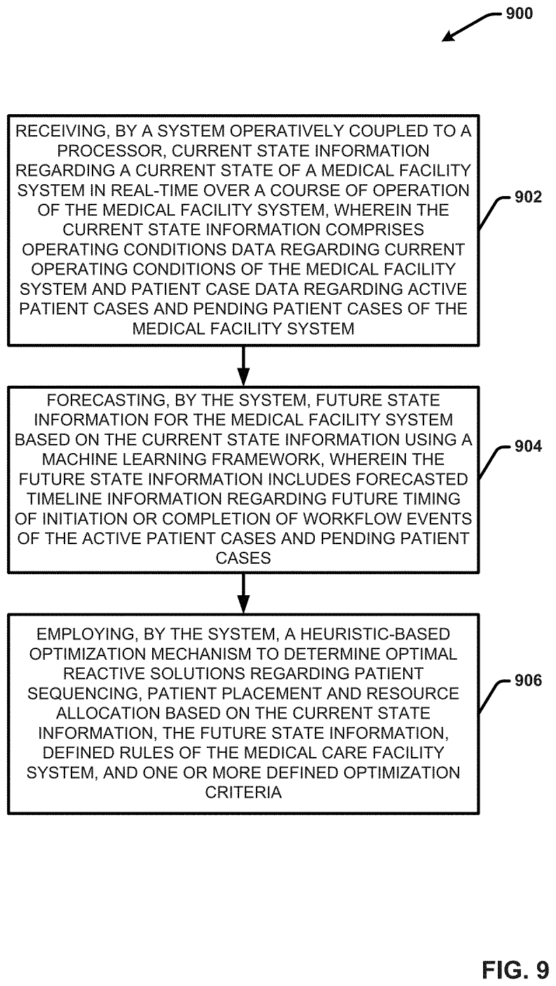

FIG. 9 illustrates an example, high-level flow diagram of a computer-implemented process 900 for optimizing the sequencing and placement of patients in a dynamic medical system in accordance with one or more embodiments of the disclosed subject matter. Repetitive description of like elements employed in other embodiments described herein is omitted for sake of brevity.

At 902, a system operatively coupled to a processor (e.g., system 100, system 600, or the like), receives (e.g., via the reception component 106) current state information (e.g., current state data 102) regarding a current state of a medical facility system in real-time over a course of operation of the medical facility system, wherein the current state information comprises operating conditions data (e.g., operating conditions data 102b) regarding current operating conditions of the medical facility system and patient case data (e.g., patient case data 102a) regarding active patient cases and pending patient cases of the medical facility system. At 904, the system forecasts future state information for the medical facility system based on the current state information using a machine learning framework (e.g., using forecasting component 108), wherein the future state information includes forecasted timeline information (e.g., timeline forecasts 136) regarding future timing of initiation or completion of workflow events of the active patient cases and pending patient cases. At 906, the system employs a heuristic-based optimization mechanism to determine (e.g., via optimization component 114) optimal reactive solutions (e.g., reactive solutions data 412) regarding patient sequencing, patient placement and resource allocation based on the current state information, the future state information, defined rules of the medical care facility system, and one or more defined optimization criteria.

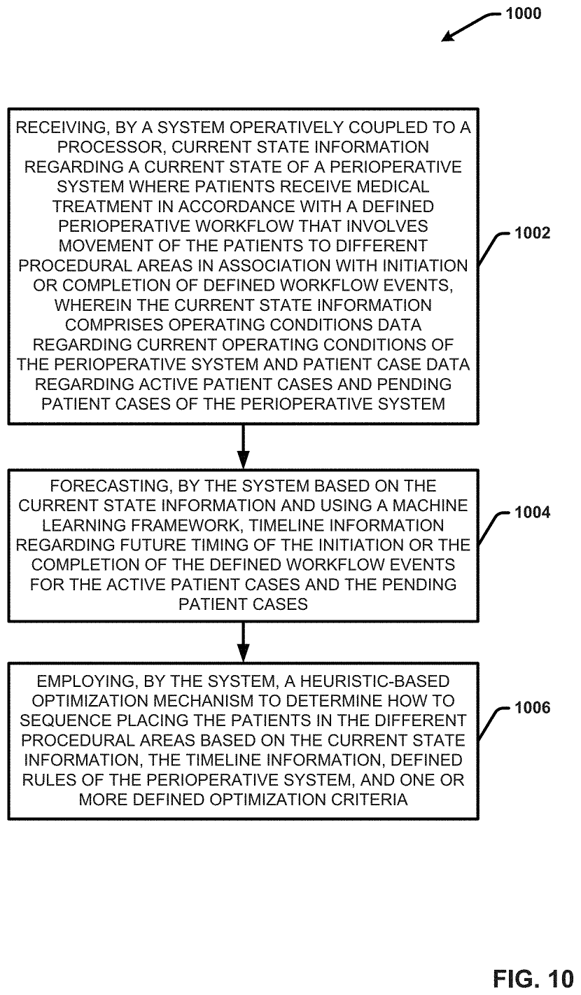

FIG. 10 illustrates another example, high-level flow diagram of a computer-implemented process 1000 for optimizing the sequencing and placement of patients in a dynamic medical system in accordance with one or more embodiments of the disclosed subject matter. Repetitive description of like elements employed in other embodiments described herein is omitted for sake of brevity.

At 1002, a system operatively coupled to a processor (e.g., system 100, system 600, or the like), receives (e.g., via the reception component 106) current state information (e.g., current state data 102) regarding a current state of a perioperative system where patients receive medical treatment in accordance with a defined perioperative workflow that involves movement of the patients to different procedural areas in association with initiation or completion of defined workflow events, wherein the current state information comprises operating conditions data (e.g., operating conditions data 102b) regarding current operating conditions of the medical facility system and patient case data (e.g., patient case data 102a) regarding active patient cases and pending patient cases of the medical facility system. At 1004, the system forecasts, based on the current state information and using a machine learning framework, timeline information (e.g., timeline forecasts 136) regarding future timing of initiation or completion of workflow events of the active patient cases and pending patient cases (e.g., via the case timeline forecasting component 110). At 1006, the system employs a heuristic-based optimization mechanism to determine (e.g., via optimization component 114) optimal reactive solutions (e.g., reactive solutions data 412) regarding patient sequencing, patient placement and resource allocation based on the current state information, the timeline information, defined rules of the perioperative system, and one or more defined optimization criteria.

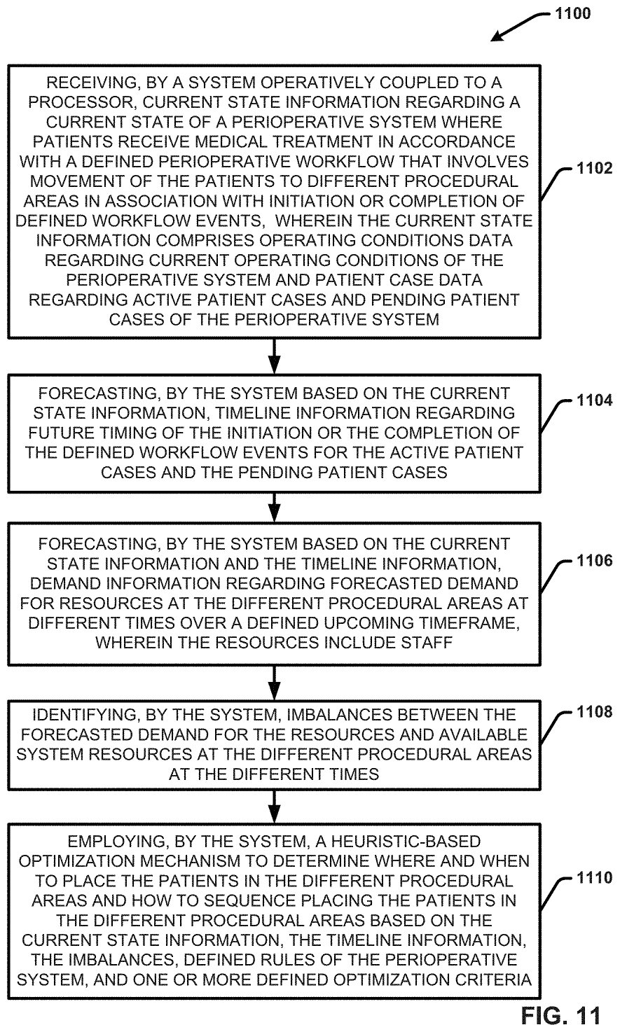

FIG. 11 illustrates another example, high-level flow diagram of a computer-implemented process 1100 for optimizing the sequencing and placement of patients in a dynamic medical system in accordance with one or more embodiments of the disclosed subject matter. Repetitive description of like elements employed in other embodiments described herein is omitted for sake of brevity.

At 1102, a system operatively coupled to a processor (e.g., system 100, system 600, or the like), receives (e.g., via the reception component 106) current state information (e.g., current state data 102) regarding a current state of a perioperative system where patients receive medical treatment in accordance with a defined perioperative workflow that involves movement of the patients to different procedural areas in association with initiation or completion of defined workflow events, wherein the current state information comprises operating conditions data (e.g., operating conditions data 102b) regarding current operating conditions of the medical facility system and patient case data (e.g., patient case data 102a) regarding active patient cases and pending patient cases of the medical facility system. At 1104, the system forecasts, based on the current state information and using a machine learning framework, timeline information (e.g., timeline forecasts 136) regarding future timing of the initiation or the completion of the defined workflow events of the active patient cases and pending patient cases (e.g., via the case timeline forecasting component 110).

At 1106, the system forecasts, based on the current state information and the timeline information, demand information (e.g., demand forecasts 138) regarding forecasted demand for resources at the different procedural areas at different times over a defined upcoming timeframe, wherein the resources include staff (e.g., via the demand forecasting component 114). At 1108, the system identifies imbalances between the forecasted demand for the resources and available system resources at the different procedural areas at the different times (e.g., via the compliance monitoring component 602). At 1110, the system employs a heuristic-based optimization mechanism to determine (e.g., via optimization component 114) where and when to place the patients in the different procedural areas and how to sequence placing the patients in the different procedural based on the current state information, the timeline information, the imbalances, defined rules of the perioperative system, and one or more defined optimization criteria.

### Example Operating Environment

One or more embodiments can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 12, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 12, an example environment 1200 for implementing various aspects of the claimed subject matter includes a computer 1202. The computer 1202 includes a processing unit 1204, a system memory 1206, a codec 1235, and a system bus 1208. The system bus 1208 couples system components including, but not limited to, the system memory 1206 to the processing unit 1204. The processing unit 1204 can be any of various available processors. Dual microprocessors and other multiprocessor architectures also can be employed as the processing unit 1204.

The system bus 1208 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 1206 includes volatile memory 1210 and non-volatile memory 1212, which can employ one or more of the disclosed memory architectures, in various embodiments. The basic input/output system (BIOS), containing the basic routines to transfer information between elements within the computer 1202, such as during start-up, is stored in non-volatile memory 1212. In addition, according to present innovations, codec 1235 can include at least one of an encoder or decoder, wherein the at least one of an encoder or decoder can consist of hardware, software, or a combination of hardware and software. Although, codec 1235 is depicted as a separate component, codec 1235 can be contained within non-volatile memory 1212. By way of illustration, and not limitation, non-volatile memory 1212 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 1212 can employ one or more of the disclosed memory devices, in at least some embodiments. Moreover, non-volatile memory 1212 can be computer memory (e.g., physically integrated with computer 1202 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 1210 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 1202 can also include removable/non-removable, volatile/non-volatile computer storage medium. FIG. 12 illustrates, for example, disk storage 1214. Disk storage 1214 includes, but is not limited to, devices like a magnetic disk drive, solid state disk (SSD), flash memory card, or memory stick. In addition, disk storage 1214 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1214 to the system bus 1208, a removable or non-removable interface is typically used, such as interface 1216. It is appreciated that disk storage 1214 can store information related to a user. Such information might be stored at or provided to a server or to an application running on a user device. In one embodiment, the user can be notified (e.g., by way of output device(s) 1236) of the types of information that are stored to disk storage 1214 or transmitted to the server or application. The user can be provided the opportunity to opt-in or opt-out of having such information collected or shared with the server or application (e.g., by way of input from input device(s) 1228).

It is to be appreciated that FIG. 12 describes software that acts as an intermediary between users and the basic computer resources described in the suitable operating environment 1200. Such software includes an operating system 1218. Operating system 1218, which can be stored on disk storage 1214, acts to control and allocate resources of the computer 1202. Applications 1220 take advantage of the management of resources by operating system 1218 through program modules 1224, and program data 1226, such as the boot/shutdown transaction table and the like, stored either in system memory 1206 or on disk storage 1214. It is to be appreciated that the claimed subject matter can be implemented with various operating systems or combinations of operating systems.

A user enters commands or information into the computer 1202 through input device(s) 1228. Input devices 1228 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1204 through the system bus 1208 via interface port(s) 1230. Interface port(s) 1230 include, for example, a serial port, a parallel port, a game port, and a universal serial bus (USB). Output device(s) 1236 use some of the same type of ports as input device(s) 1228. Thus, for example, a USB port can be used to provide input to computer 1202 and to output information from computer 1202 to an output device 1236. Output adapter 1234 is provided to illustrate that there are some output devices 1236 like monitors, speakers, and printers, among other output devices 1236, which require special adapters. The output adapters 1234 include, by way of illustration and not limitation, video and sound cards that provide a means of connection between the output device 1236 and the system bus 1208. It should be noted that other devices or systems of devices provide both input and output capabilities such as remote computer(s) 1238.

Computer 1202 can operate in a networked environment using logical connections to one or more remote computers, such as remote computer(s) 1238. The remote computer(s) 1238 can be a personal computer, a server, a router, a network PC, a workstation, a microprocessor based appliance, a peer device, a smart phone, a tablet, or other network node, and typically includes many of the elements described relative to computer 1202. For purposes of brevity, only a memory storage device 1240 is illustrated with remote computer(s) 1238. Remote computer(s) 1238 is logically connected to computer 1202 through a network interface 1242 and then connected via communication connection(s) 1244. Network interface 1242 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1244 refers to the hardware/software employed to connect the network interface 1242 to the bus 1208. While communication connection 1244 is shown for illustrative clarity inside computer 1202, it can also be external to computer 1202. The hardware/software necessary for connection to the network interface 1242 includes, for exemplary purposes only, internal and external technologies such as, modems including regular telephone grade modems, cable modems and DSL modems, ISDN adapters, and wired and wireless Ethernet cards, hubs, and routers.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a memory that stores computer executable components; and
a processor that executes the computer executable components stored in the memory, wherein the computer executable components comprise:
a reception component that receives current state information regarding a current state of a medical facility system in real-time over a course of operation of the medical facility system, wherein the current state information comprises operating conditions data regarding current operating conditions of the medical facility system and patient case data regarding active patient cases and pending patient cases of the medical facility system;
a forecasting component that employs a machine learning framework to forecast future state information for the medical facility system based on the current state information, wherein the future state information includes forecasted timeline information regarding future timing of initiation or completion of workflow events of the active patient cases and the pending patient cases; and
an optimization component that employs a heuristic-based optimization mechanism to determine optimal reactive solutions regarding patient sequencing, patient placement and resource allocation based on the current state information, the future state information, defined rules of the medical care facility system, and one or more defined optimization criteria.

2. The system of claim 1, wherein the forecasting component employs the machine learning framework to repeatedly forecast and update the future state information based on the current state information as the current state information is received, and wherein the optimization component employs the heuristic-based optimization mechanism to repeatedly determine and update the optimal reactive solutions based on the current state information as the current state information is received and the future state information as it is forecasted and updated.

3. The system of claim 2, wherein the computer executable components further comprise:
a reporting component that provides the future state information and solutions information regarding the optimal reactive solutions as they are respectively forecasted and determined in real-time to one or more entities at the medical care facility system to facilitate managing operations of the medical care facility.

4. The system of claim 3, wherein the computer executable components further comprise:
a display component that displays the future state information and the solutions information in real-time via a graphical user interface tile at one or more devices associated with one or more entities.

5. The system of claim 1, wherein the medical facility system comprises a perioperative system where patients receive medical treatment in accordance with a defined perioperative workflow that involves movement of the patients to different procedural areas in association with the initiation or the completion of the workflow events, and wherein the optimization component determines how to sequence placing the patients in the different procedural areas in accordance with the defined perioperative workflow.

6. The system of claim 5, wherein the one or more defined optimization criteria comprises minimizing delays between the workflow events and initiation of the pending patient cases.

7. The system of claim 5, wherein the one or more defined optimization criteria are selected from a group consisting of: minimizing blocking of patients to required treatment, maximizing patient flow, minimizing costs, and minimizing surgeon wait time between procedures.

8. The system of claim 5, wherein the patient case data comprises status tracking information for the active patient cases indicating a current status of the active patient cases relative to the defined perioperative workflow and wherein the forecasting component determines the forecasted timeline information based on the current status of the active patient cases and historical state information regarding historical timing of the workflow events for historical patient cases under varying operating conditions of the perioperative system.

9. The system of claim 8, wherein the forecasting component comprises a case timeline forecasting component that determines the forecasted timeline information using one or more machine learning models trained on the historical state information, and wherein the case timeline forecasting component regularly updates the one or more machine learning models based on the current state information.

10. The system of claim 5, wherein the current operating conditions data comprises bed status information identifying availability status of beds in the different procedural areas and staffing information identifying staff assigned to the beds, and wherein the optimization component determines how to sequence placing the patients in the different procedural areas based on the forecasted timeline information, the availability status of the beds, the staff assigned to the beds, first defined rules regarding types of beds where the patients can be placed, and second defined rules regarding preferred staff to patient ratios in the different procedural areas.

11. The system of claim 5, wherein the future state information further includes forecasted demand information regarding forecasted demand for resources at the different procedural areas at different times over a defined upcoming timeframe, and wherein the resources include staff.

12. The system of claim 11, wherein the forecasting component comprises a demand forecasting component that determines the forecasted demand information based on the current state information and the forecasted timeline information.

13. The system of claim 11, wherein the computer executable components further comprise:
a compliance monitoring component that determines whether available system resources at the different procedural areas at the different times comply with a resource constraint for the perioperative system based on forecasted demand; and
an alert component that generates an alert regarding failure of the available system resources to comply with the resource constraint based on determination that the available system resources at a procedural area of the different procedural areas at a time of the different times, fail to comply with the resource constraint.

14. The system of claim 11, wherein the computer executable components further comprise:
a compliance monitoring component that identifies imbalances between the forecasted demand for the resources and available system resources at the different procedural areas at the different times, and wherein the optimization component further employs the heuristic-based optimization mechanism to determine the optimal reactive solutions based on the imbalances.

15. A method, comprising:
receiving, by a system operatively coupled to a processor, current state information regarding a current state of a medical facility system in real-time over a course of operation of the medical facility system, wherein the current state information comprises operating conditions data regarding current operating conditions of the medical facility system and patient case data regarding active patient cases and pending patient cases of the medical facility system;
forecasting, by the system, future state information for the medical facility system based on the current state information using a machine learning framework, wherein the future state information includes forecasted timeline information regarding future timing of initiation or completion of workflow events of the active patient cases and the pending patient cases; and
employing, by the system, a heuristic-based optimization mechanism to determine optimal reactive solutions regarding patient sequencing, patient placement and resource allocation based on the current state information, the future state information, defined rules of the medical care facility system, and one or more defined optimization criteria.

16. The method of claim 15, wherein the forecasting comprises repeatedly forecasting and updating the future state information based on the current state information as the current state information is received, and wherein the employing comprises employing the heuristic-based optimization mechanism to repeatedly determine and update the optimal reactive solutions based on the current state information as the current state information is received and the future state information as it is forecasted and updated.

17. The method of claim 16, further comprising:
providing, by the system via a display tile of a graphical user interface, the future state information and solutions information regarding the optimal reactive solutions as they are respectively forecasted and determined in real-time to one or more entities at the medical care facility system to facilitate managing operations of the medical care facility.

18. The method of claim 15, wherein the medical facility system comprises a perioperative system where patients receive medical treatment in accordance with a defined perioperative workflow that involves movement of the patients to different procedural areas in association with the initiation or the completion of the workflow events, and wherein employing comprises employing the heuristic-based optimization mechanism to determine how to sequence placing the patients in the different procedural areas in accordance with the defined perioperative workflow.

19. The method of claim 18, wherein the one or more defined optimization criteria comprises minimizing delays between the workflow events and initiation of the pending patient cases.

20. The method of claim 18, wherein the patient case data comprises status tracking information for the active cases indicating a current status of the active patient cases relative to the defined perioperative workflow, and wherein the forecasting comprises determining the forecasted timeline information based on the current status of the active patient cases and historical state information regarding historical timing of the workflow events for historical patient cases under varying operating conditions of the perioperative system.

21. The method of claim 18, wherein the future state information further includes forecasted demand information regarding forecasted demand for resources at the different procedural areas at different times over a defined upcoming timeframe, and wherein the resources include staff.

22. The method of claim 21, wherein the forecasting comprises determining the forecasted demand information based on the current state information and the forecasted timeline information.

23. The method of claim 21, further comprising:
determining, by the system, whether available system resources at the different procedural areas at the different times comply with a resource constraint for the perioperative system based on forecasted demand; and
generating, by the system, an alert regarding failure of the available system resources to comply with the resource constraint based on determination that the available system resources at a procedural area of the different procedural areas at a time of the different times, fail to comply with the resource constraint.

24. The method of claim 21, further comprising:
identifying, by the system, imbalances between the forecasted demand for the resources and available system resources at the different procedural areas at the different times, and wherein the employing further comprises employing the heuristic-based optimization mechanism to determine the optimal reactive solutions based on the imbalances.

25. A machine-readable storage medium, comprising executable instructions that, when executed by a processor, facilitate performance of operations, comprising:
receiving current state information regarding a current state of a perioperative system where patients receive medical treatment in accordance with a defined perioperative workflow that involves movement of the patients to different procedural areas in association with initiation or completion of defined workflow events, wherein the current state information comprises operating conditions data regarding current operating conditions of the perioperative system and patient case data regarding active patient cases and pending patient cases of the perioperative system;
forecasting based on the current state information and using a machine learning framework, timeline information regarding future timing of the initiation or the completion of the defined workflow events for the active patient cases and the pending patient cases; and
employing a heuristic-based optimization mechanism to determine how to sequence placing the patients in the different procedural areas based on the current state information, the timeline information, defined rules of the perioperative system, and one or more defined optimization criteria.
